SEQUENCE LISTING (143 sequences)

SEQ ID NO: 1 - DNA, 16 bp, Homo sapiens
caggtcttggtaggtg

SEQ ID NO: 2 - DNA, 16 bp, Homo sapiens
agaggactatgagacc

SEQ ID NO: 3 - DNA, 16 bp, Homo sapiens
agcgctcgccacgctc

SEQ ID NO: 4 - DNA, 16 bp, Homo sapiens
cccctaaaatatttct

SEQ ID NO: 5 - DNA, 16 bp, Homo sapiens
ttttcagtgagcgtgg

SEQ ID NO: 6 - DNA, 17 bp, Homo sapiens
ggcccgcgcccctgttt

SEQ ID NO: 7 - DNA, 17 bp, Homo sapiens
tgtggatcccacccaaa

SEQ ID NO: 8 - DNA, 16 bp, Homo sapiens
tgattggtgcccagtt

SEQ ID NO: 9 - DNA, 16 bp, Homo sapiens
ggtgccaccaacccat

SEQ ID NO: 10 - DNA, 16 bp, Homo sapiens
aactcggtggctgcct

SEQ ID NO: 11 - DNA, 16 bp, Homo sapiens
ctaggtgagcgcattg

SEQ ID NO: 12 - DNA, 17 bp, Homo sapiens
cgcactctttacggctt

SEQ ID NO: 13 - DNA, 16 bp, Homo sapiens
aatgatgccctggaga

SEQ ID NO: 14 - DNA, 16 bp, Homo sapiens
aggactttgtgaagga

SEQ ID NO: 15 - DNA, 17 bp, Homo sapiens
ctattaacgctacgatg

SEQ ID NO: 16 - DNA, 17 bp, Homo sapiens
gacttccactgccatcc

SEQ ID NO: 17 - DNA, 16 bp, Homo sapiens
tcacccaaaccggcag

SEQ ID NO: 18 - DNA, 16 bp, Homo sapiens
ctacatggctgcggtc

SEQ ID NO: 19 - DNA, 16 bp, Homo sapiens
acctgaagcagcaagt

SEQ ID NO: 20 - DNA, 17 bp, Homo sapiens
cgactttcccgatcgcc

SEQ ID NO: 21 - DNA, 16 bp, Homo sapiens
gccgagagccacactt

SEQ ID NO: 22 - DNA, 20 bp, Homo sapiens
gatagtcccttgaacacaca

SEQ ID NO: 23 - DNA, 16 bp, Homo sapiens
ggcccgcgtctcgaag

SEQ ID NO: 24 - DNA, 16 bp, Homo sapiens
gagaccgggtgttcct

SEQ ID NO: 25 - DNA, 16 bp, Homo sapiens
gctgtgcaccctagaa

SEQ ID NO: 26 - DNA, 16 bp, Homo sapiens
cgcccagggcggcgtt

SEQ ID NO: 27 - DNA, 17 bp, Homo sapiens
atccttcctgtttactc

SEQ ID NO: 28 - DNA, 17 bp, Homo sapiens
tttcagctcttgacctg

SEQ ID NO: 29 - DNA, 16 bp, Homo sapiens
gtggcacctggaattt

SEQ ID NO: 30 - DNA, 16 bp, Homo sapiens
cagtgctcattttgag

SEQ ID NO: 31 - DNA, 16 bp, Homo sapiens
acaggcctttgacctc

SEQ ID NO: 32 - DNA, 17 bp, Homo sapiens
taccttcagcctccatc

SEQ ID NO: 33 - DNA, 16 bp, Homo sapiens
gggtttaggaaacgtg

SEQ ID NO: 34 - DNA, 17 bp, Homo sapiens
tcctccccacctaggcc

SEQ ID NO: 35 - DNA, 16 bp, Homo sapiens
gtcagctggatgacca

SEQ ID NO: 36 - DNA, 17 bp, Homo sapiens
tcctggtgtcacgccct

SEQ ID NO: 37 - DNA, 16 bp, Homo sapiens
gatccgctaaggggca

SEQ ID NO: 38 - DNA, 16 bp, Homo sapiens
aggagggaaccccgtt

SEQ ID NO: 39 - DNA, 16 bp, Homo sapiens
tactccaggccaaagg

SEQ ID NO: 40 - DNA, 17 bp, Homo sapiens
gccatcgtgtgcttgtt

SEQ ID NO: 41 - DNA, 15 bp, Homo sapiens
tccagcgcaaggagg

SEQ ID NO: 42 - DNA, 17 bp, Homo sapiens
gctgtggctccttccta

SEQ ID NO: 43 - DNA, 16 bp, Homo sapiens
catgtctggacctcag

SEQ ID NO: 44 - DNA, 17 bp, Homo sapiens
ccctcatgaacccaaca

SEQ ID NO: 45 - DNA, 16 bp, Homo sapiens
gcagcctgtgctgagg

SEQ ID NO: 46 - DNA, 16 bp, Homo sapiens
ggccttgagacttgaa

SEQ ID NO: 47 - DNA, 16 bp, Homo sapiens
ttggttttgagaccac

SEQ ID NO: 48 - DNA, 16 bp, Homo sapiens
ggctttgcaggggctc

SEQ ID NO: 49 - DNA, 16 bp, Homo sapiens
ggtggtcactgggaat

SEQ ID NO: 50 - DNA, 17 bp, Homo sapiens
gtatgctctatttcctg

SEQ ID NO: 51 - DNA, 16 bp, Homo sapiens
agtttcagtaggtcat

SEQ ID NO: 52 - DNA, 17 bp, Homo sapiens
cgctgtactttgctgac

SEQ ID NO: 53 - DNA, 17 bp, Homo sapiens
gttctgattttcttgta

SEQ ID NO: 54 - DNA, 17 bp, Homo sapiens
ggtcactgattttccca

SEQ ID NO: 55 - DNA, 16 bp, Homo sapiens
ccaacagagtctaggc

SEQ ID NO: 56 - DNA, 16 bp, Homo sapiens
cgccatcacagctgaa

SEQ ID NO: 57 - DNA, 16 bp, Homo sapiens
gagctctgggcccttc

SEQ ID NO: 58 - DNA, 17 bp, Homo sapiens
cacttaccacctcattt

SEQ ID NO: 59 - DNA, 16 bp, Homo sapiens
aaccccacagccttga

SEQ ID NO: 60 - DNA, 16 bp, Homo sapiens
cagattacccaaaagg

SEQ ID NO: 61 - DNA, 17 bp, Homo sapiens
cctctcaacacacagca

SEQ ID NO: 62 - DNA, 17 bp, Homo sapiens
cctcactgagtcatcag

SEQ ID NO: 63 - DNA, 17 bp, Homo sapiens
ccctcccatgccgttcc

SEQ ID NO: 64 - DNA, 19 bp, Homo sapiens
tgacatcagcctgcttctt

SEQ ID NO: 65 - DNA, 16 bp, Homo sapiens
tagggagtagaaccgt

SEQ ID NO: 66 - DNA, 16 bp, Homo sapiens
ctctggtctgagtcaa

SEQ ID NO: 67 - DNA, 17 bp, Homo sapiens
cttctgttgcctccaga

SEQ ID NO: 68 - DNA, 16 bp, Homo sapiens
gtggctaagagcccaa

SEQ ID NO: 69 - DNA, 16 bp, Homo sapiens
gccagccttcctcaag

SEQ ID NO: 70 - DNA, 16 bp, Homo sapiens
ggctcccaaggccatg

SEQ ID NO: 71 - DNA, 16 bp, Homo sapiens
ccgtgatgtacacgta

SEQ ID NO: 72 - DNA, 16 bp, Homo sapiens
atgctaactagttacg

SEQ ID NO: 73 - DNA, 16 bp, Homo sapiens
gtcagaaccttggatt

SEQ ID NO: 74 - DNA, 16 bp, Homo sapiens
cacccctggaagatgg

SEQ ID NO: 75 - DNA, 16 bp, Homo sapiens
gaaatgtattgtttga

SEQ ID NO: 76 - DNA, 17 bp, Homo sapiens
gactttgctgttctact

SEQ ID NO: 77 - DNA, 16 bp, Homo sapiens
gctggagcccgcaccc

SEQ ID NO: 78 - DNA, 16 bp, Homo sapiens
ctgggcacagagaatc

SEQ ID NO: 79 - DNA, 16 bp, Homo sapiens
gtagattcccaagaga

SEQ ID NO: 80 - DNA, 16 bp, Homo sapiens
tgcgccgacgatgccc

SEQ ID NO: 81 - DNA, 16 bp, Homo sapiens
ctcctggtgggacttg

SEQ ID NO: 82 - DNA, 17 bp, Homo sapiens
ccagttcttcctgacac

SEQ ID NO: 83 - DNA, 16 bp, Homo sapiens
tctggacgtggtaagc

SEQ ID NO: 84 - DNA, 16 bp, Homo sapiens
cacaaatcccctgcaa

SEQ ID NO: 85 - DNA, 17 bp, Homo sapiens
ctaggctttgcttagta

SEQ ID NO: 86 - DNA, 16 bp, Homo sapiens
gggagtcacttgatgc

SEQ ID NO: 87 - DNA, 17 bp, Homo sapiens
ttcttgtgaaatgtgtc

SEQ ID NO: 88 - DNA, 15 bp, Homo sapiens
ctgggaaccgggagt

SEQ ID NO: 89 - DNA, 16 bp, Homo sapiens
atccaggtgtcaagaa

SEQ ID NO: 90 - DNA, 16 bp, Homo sapiens
cccactggggggttgg

SEQ ID NO: 91 - DNA, 16 bp, Homo sapiens
tccaggaatagcatat

SEQ ID NO: 92 - DNA, 16 bp, Homo sapiens
cacaggtccatgaaag

SEQ ID NO: 93 - DNA, 16 bp, Homo sapiens
cagcacttgtgaagga

SEQ ID NO: 94 - DNA, 16 bp, Homo sapiens
gtgggctaagataggt

SEQ ID NO: 95 - DNA, 17 bp, Homo sapiens
gccctttttccttctaa

SEQ ID NO: 96 - DNA, 17 bp, Homo sapiens
gcgccatcttgccctgt

SEQ ID NO: 97 - DNA, 16 bp, Homo sapiens
ctccaccatgggaatc

SEQ ID NO: 98 - DNA, 16 bp, Homo sapiens
cgggggccttccctcg

SEQ ID NO: 99 - DNA, 15 bp, Homo sapiens
ggaagagcaagactg

SEQ ID NO: 100 - DNA, 17 bp, Homo sapiens
tgtcttgggaatgtttc

SEQ ID NO: 101 - DNA, 17 bp, Homo sapiens
gagtctgtttgctgtta

SEQ ID NO: 102 - DNA, 17 bp, Homo sapiens
cttccatctggctgcac

SEQ ID NO: 103 - DNA, 15 bp, Homo sapiens
atccaggcgggagca

SEQ ID NO: 104 - DNA, 16 bp, Homo sapiens
gaagagttgcaaaatg

SEQ ID NO: 105 - DNA, 16 bp, Homo sapiens
cagacagagcccactt

SEQ ID NO: 106 - DNA, 15 bp, Homo sapiens
cgcagatggagggac

SEQ ID NO: 107 - DNA, 16 bp, Homo sapiens
ccctgtagtccagtgg

SEQ ID NO: 108 - DNA, 16 bp, Homo sapiens
gatccacattgttagg

SEQ ID NO: 109 - DNA, 16 bp, Homo sapiens
ccaagtcagcagtcct

SEQ ID NO: 110 - DNA, 17 bp, Homo sapiens
tgcctggctgtctcctt

SEQ ID NO: 111 - DNA, 16 bp, Homo sapiens
ctgctctagaaagtat

SEQ ID NO: 112 - DNA, 16 bp, Homo sapiens
gctgcatggcagatgc

SEQ ID NO: 113 - DNA, 16 bp, Homo sapiens
ggccattctggtcatc

SEQ ID NO: 114 - DNA, 17 bp, Homo sapiens
gttggcaccttcgtttc

SEQ ID NO: 115 - DNA, 16 bp, Homo sapiens
gtgcggatttgtgact

SEQ ID NO: 116 - DNA, 50 bp, Homo sapiens
agatatttccccagaggcacgcgaactgtcagtctttcctaaggcccccg

SEQ ID NO: 117 - DNA, 50 bp, Homo sapiens
gcaaggtaaaacgcacagggttcccttatgtagatgtacacatggcattg

SEQ ID NO: 118 - DNA, 50 bp, Homo sapiens
aagcctacctgtggaaggaaagtttctcttccaaataaagccttagaatt

SEQ ID NO: 119 - DNA, 50 bp, Homo sapiens
ggatgagagggaaccactataacatgagtccaagcccagaagacttctgt

SEQ ID NO: 120 - DNA, 50 bp, Homo sapiens
gtgacggctggtgactgatggatgggtagtgggctgagaagaggggacta

SEQ ID NO: 121 - DNA, 50 bp, Homo sapiens
tcctttaggaatgctgtgtgtctgtccggcaccccggtaggcctgattgg

SEQ ID NO: 122 - DNA, 50 bp, Homo sapiens
cagcatgatccctcagctgaactcaaaagccgtcaaagaccttgcctccc

SEQ ID NO: 123 - DNA, 50 bp, Homo sapiens
gaatcagcttccaggtgtagggaccccttgaggggccaagctgacatcca

SEQ ID NO: 124 - DNA, 50 bp, Homo sapiens
cttccgctaggcccatcgtttctcaggctgcccaaggctcttctgtaaca

SEQ ID NO: 125 - DNA, 50 bp, Homo sapiens
ggtagcctagttatttgagcctggtttcaatgtgagaaccacgtttactg

SEQ ID NO: 126 - DNA, 50 bp, Homo sapiens
tgatggattcttccatcctgccggatgagtgtgggtgtgatacagcctac

SEQ ID NO: 127 - DNA, 50 bp, Homo sapiens
tcaatgtgtatgctctgtccccatccttcactcctcctcaagctcacacc

SEQ ID NO: 128 - DNA, 50 bp, Homo sapiens
gttgcatttcatgggcctgggggtttcctagcagaggatattggagcccc

SEQ ID NO: 129 - DNA, 50 bp, Homo sapiens
cctttgcacagcatccttggaggggattcgcaagtggcagccctgttgat

SEQ ID NO: 130 - DNA, 50 bp, Homo sapiens
aaggccagtgcagccaaaagcagcctcatccaccaaggcatccgctgaag

SEQ ID NO: 131 - DNA, 50 bp, Homo sapiens
tccctgaacgacactctcctaatcctaagccttacctgagtgagaagccc

SEQ ID NO: 132 - DNA, 50 bp, Homo sapiens
gctgtgttccccaactctgttctgcgcacgaaacagtatctgtttgaggc

SEQ ID NO: 133 - DNA, 50 bp, Homo sapiens
agaaggattatgaggaggttggagcagatagtgctgacggagaggatgag

SEQ ID NO: 134 - DNA, 50 bp, Homo sapiens
acccagtgttttaggagcatgagtgccgtgtgtgtgcgtcctgtcggagc

SEQ ID NO: 135 - DNA, 50 bp, Homo sapiens
cagagccccagcccctcatgtcttgccgcccttcctccatgtgtttgtaa

SEQ ID NO: 136 - DNA, 50 bp, Homo sapiens
ccctcaaagtgtcacaatgctcctcctgatgactcctccccagaaaacca

SEQ ID NO: 137 - DNA, 50 bp, Homo sapiens
gggcagattctggctgccttttaatttcgtccttcacctgatatctgtgc

SEQ ID NO: 138 - DNA, 50 bp, Homo sapiens
aaagcagatatttcccggacccagcgcggcctcaaccagggcaggaaaga

SEQ ID NO: 139 - DNA, 50 bp, Homo sapiens
ctggctgaagcatccccttggagtgccatgtataagttgggctattagag

SEQ ID NO: 140 - DNA, 50 bp, Homo sapiens
tgaatgaagccctggagtactatgagcgggccctgagactggctgctgac

SEQ ID NO: 141 - DNA, 50 bp, Homo sapiens
acgcagccagaatccttaagtctgtgtgtttctgtgtctcaagactgggc

SEQ ID NO: 142 - DNA, 50 bp, Homo sapiens
ccaacatgctctaatgcttcagattcaagtgctttttccactgtttcccc

SEQ ID NO: 143 - DNA, 50 bp, Homo sapiens
ttgcccaccccgcacagcctgagtttgcaataaaactgggacactgggac

(19) United States
(12) Patent Application Publication
(10) Pub. No.: US 2013/0295581 A1
(43) Pub. Date: Nov. 7, 2013

(54) Methods and Compositions for the Treatment and Diagnosis of Breast Cancer

(71) Applicant: Oncocyte Corporation, Alameda, CA (US)

(72) Inventors: Karen Chapman, Mill Valley, CA (US); Joseph Wagner, San Ramon, CA (US); Michael West, Mill Valley, CA (US)

(21) Appl. No.: 13/888,254

(22) Filed: May 6, 2013

Related U.S. Application Data
(60) Provisional application No. 61/643,190, filed on May 4, 2012.

(51) Int. Cl. G01N 33/68

(52) U.S. Cl. CPC G01N 33/6893
USPC 435/6.14; 435/7.92

(57) ABSTRACT

The invention provides methods, compositions and kits relating to the diagnosis, prognosis and treatment of breast cancer.

This application claims priority to U.S. Provisional Application No. 61/643,190 filed May 4, 2012, the entire contents of which is hereby incorporated by reference.

### FIELD OF THE INVENTION

The field of the invention relates to cancer and the diagnosis and treatment of cancer.

### BACKGROUND

Early detection of cancer can impact treatment outcomes and disease progression. Typically, cancer detection relies on diagnostic information obtained from biopsy, x-rays, CAT scans, NMR and the like. These procedures may be invasive, time consuming and expensive. Moreover, they have limitations with regard to sensitivity and specificity. There is a need in the field of cancer diagnostics for a highly specific, highly sensitive, rapid, inexpensive, and relatively non-invasive method of diagnosing cancer. Various embodiments of the invention described below meet this need as well as other needs in the field of diagnosing and treating cancer.

### SUMMARY OF THE INVENTION

Embodiments of the disclosure provide for methods of diagnosis, prognosis and treatment of breast cancer.

In certain embodiments the invention provides a method of detecting breast cancer in a subject comprising a) contacting a sample obtained from the subject with one or more agents that detect one or more markers expressed by a breast cancer cell b) contacting a non-cancerous cell with the one or more agents from a); and c) comparing the expression level of the marker in the sample obtained from the subject with the expression level in the non-cancerous cell, wherein a higher level of expression of the marker in the sample compared to the non-cancerous cell indicates that the subject has breast cancer.

In certain embodiments the invention provides a method of detecting breast cancer in a subject comprising a) contacting a sample obtained from the subject with one or more agents that detect expression of at least one of the markers listed in Table 1 and or Table 2; b) contacting a non-cancerous cell, e.g. a non-cancerous cell from breast tissue, with the one or more agents from a); and c) comparing the expression level of one or more of the markers listed in Table 1 in the sample obtained from the subject with the expression level of one or more of the markers listed in Table 1 and/or Table 2 in the non-cancerous cell, wherein a higher level of expression of one or more of the markers listed in Table 1 in the sample compared to the non-cancerous cell indicates that the subject has breast cancer.

In some embodiments the invention provides a method of detecting breast cancer in a subject comprising a) contacting a sample obtained from the subject with one or more agents that detect expression of at least one of the markers chosen from FSIP1, CXCL9; MMP7; MMP13 and MMP12; b) contacting a non-cancerous cell, e.g. a non-cancerous cell from breast tissue, with the one or more agents from a); and c) comparing the expression revel of one or more of the markers chosen from FSIP1, CXCL9, MMP7, MMP13 and MMP12 in the sample obtained from the subject with the expression level of one or more of the markers chosen from FSIP1, CXCL9, MMP7, MMP13 and MMP12 in the non-cancerous cell, wherein a higher level of expression of one or more of the markers chosen from FSIP1, CXCL9, MMP7, MMP13 and MMP12 in the sample compared to the non-cancerous cell indicates that the subject has breast cancer.

With regard to the embodiments described in the preceding paragraphs, the sample may be any sample as described infra, for example, a bodily fluid, such as blood, serum or urine. The sample may be a cellular sample or the extract of a cellular sample. The agent may be one or more molecules that bind specifically to one or more proteins expressed by the cancer cell or one or more nucleic acids expressed by the cell. For example, the agent may be a ptoein such as an antibody that binds specifically to the protein expressed by one of the marker genes identified infra. The agent may be one or more nucleic acids that hybridize to a nucleic acid expressed by the cancer cell. The nucleic acid expressed by the cancer cell may be an RNA molecule, e.g. an mRNA molecule. The nucleic acid molecule that hybridizes to the nucleic acid expressed by the cancer cell may be a DNA molecule, such as a DNA probe.

In still other embodiments the invention provides a composition of matter useful in distinguishing a breast cancer cell from a non-cancerous cell comprising one or more molecules that specifically bind to a molecule expressed at higher levels on a breast cancer cell compared to a non-cancer cell. As an example, the composition may comprise a protein, that binds to one or more molecules expressed by the cancer cell at higher levels compared to the non-cancer cell. As another example, the composition may comprise a nucleic acid that binds to one or more molecules expressed by the breast cancer cell at higher levels compared to the non-cancer cell.

In some embodiments the invention provides a composition of matter comprising a protein, such as an antibody, that specifically binds to a molecule expressed by a breast cancer cell chosen from the markers listed in Table 1 and/or Table 2. The molecule expressed by the breast cancer cell may be expressed by the breast cancer cell at level that is higher than the level expressed by a non-cancerous cell such as a non-cancerous breast tissue cell.

In certain embodiments the invention provides a composition of matter comprising a protein, such as an antibody, that specifically binds to a molecule expressed by a breast cancer cell chosen from FSIP1, CXCL9, MMP7, MMP13 and MMP12. The molecule expressed by the breast cancer cell may be expressed by the breast cancer cell at level that is higher than the level expressed by a non-cancerous cell such as a non-cancerous breast tissue cell.

In other embodiments the invention provides a composition of matter comprising a nucleic acid that specifically binds to a molecule, such as an mRNA molecule, expressed by a breast cancer cell wherein the molecule is chosen from a marker listed in Table 1 and/or Table 2. The molecule expressed by the breast cancer cell may be expressed by the breast cancer cell at level that is higher than the level expressed by a non-cancerous cell such as a non-cancerous breast tissue cell.

In other embodiments the invention provides a composition of matter comprising a nucleic acid that specifically binds to a molecule, such as an mRNA molecule, expressed by a breast cancer cell wherein the molecule is chosen from FSIP1, CXCL9, MMP7, MMP13 and MMP12. The molecule expressed by the breast cancer cell may be expressed by the breast cancer cell at level that is higher than the level expressed by a non-cancerous cell such as a non-cancerous breast tissue cell.

In still further embodiments the invention provides a method of determining if a cancer in a subject is advancing comprising a) measuring the expression level of one or more markers associated with cancer at a first time point; b) measuring the expression level of the one or more markers measured in a) at a second time point, wherein the second time point is subsequent to the first time point; and c) comparing the expression level measured in a) and b), wherein an increase in the expression level of the one or more markers in b) compared to a) indicates that the subject's cancer is advancing.

In some embodiments the invention provides a method of determining if a breast cancer in a subject is advancing comprising a) measuring the expression level of one or more markers listed in Table 1 and/or Table 2 at a first time point; b) measuring the expression level of the one or more markers measured in a) at a second time point, wherein the second time point is subsequent to the first time point; and c) comparing the expression level measured in a) and b), wherein an increase in the expression level of the one or more markers at the second time point compared to the first time point indicates that the subject's breast cancer is advancing.

In other embodiments the invention provides a method of determining if a breast cancer in a subject is advancing comprising a) measuring the expression level of one or more markers chosen from FSIP1, CXCL9, MMP7, MMP13 and MMP12 at a first time point; b) measuring the expression level of the one or more markers measured in a) at a second time point, wherein the second time point is subsequent to the first time point; and c) comparing the expression level measured in a) and b), wherein an increase in the expression level of the one or more markers at the second time point compared to the first time point indicates that the subject's breast cancer is advancing.

In some embodiments the invention provides antigens (i.e. cancer-associated polypeptides) associated with breast cancer as targets for diagnostic and/or therapeutic antibodies. In some embodiments, the antigen may be chosen from a protein encoded by, a gene listed in Table 1 and/or Table 2, a fragment thereof, or a combination of proteins encoded by a gene listed in Table 1 and/or Table 2.

In some embodiments the invention provides antigens (i.e. cancer-associated polypeptides) associated with breast cancer as targets for diagnostic and/or therapeutic antibodies. In some embodiments, the antigen may be chosen from a protein encoded by, a gene chosen from FSIP1, CXCL9, MMP7, MMP13 and MMP12, a fragment thereof, or a combination of proteins encoded by a gene chosen from FSIP1, CXCL9, MMP7, MMP13 and MMP12.

In yet other embodiments the invention provides a method of eliciting an immune response to a breast cancer cell comprising contacting a subject with a protein or protein fragment that is expressed by a breast cancer cell thereby eliciting an immune response to the cancer cell. As an example the subject may be contacted intravenously or intramuscularly.

In further embodiments the invention provides a method of eliciting an immune response to a breast cancer cell comprising contacting a subject with one or more proteins or protein fragments that is encoded by a gene chosen from the genes listed in Table 1 and/or Table 2, thereby eliciting an immune response to a breast cancer cell. As an example the subject may be contacted intravenously or intramuscularly.

In still other embodiments the invention provides a method of eliciting an immune response to a breast cancer cell comprising contacting a subject with one or more proteins or protein fragments that is encoded by a gene chosen from FSIP1, CXCL9, MMPI, MMP13 and MMP12, thereby eliciting an immune response to a breast cancer cell. As an example the subject may be contacted intravenously or intramuscularly.

In other embodiments the invention provides a kit for detection of cancer in a sample obtained from a subject. The kit may comprise one or more agents that bind specifically to a molecule expressed specifically by a breast cancer cell, e.g. one or more of the genes listed in Tables 1 and/or Table 2 and/or one or more of the proteins encoded for by one or more of the genes listed in Table 1 and/or Table 2. The kit may comprise one or more containers and instructions for determining if the sample is positive for cancer. The kit may optionally contain one or more multiwell plates, a detectable substance such as a dye, a radioactively labeled molecule, a chemiluminescently labeled molecule and the like. The kit may further contain a positive control (e.g. one or more cancerous breast cells; or specific known quantities of the molecule expressed by the cancer cell) and a negative control (e.g. a tissue or cell sample that is non-cancerous).

In some embodiments the invention provides a kit comprising a plurality of agents that bind to a plurality of genes listed in Tables 1 and/or Table 2. As an example the kit may provide a plurality of probes, e.g. nucleic acid probes, that bind to a plurality of genes found in Table 1 and/or Table. 2. In other embodiments the invention provides a kit comprising a plurality of agents that bind to a protein encoded for by a plurality of genes found in Table 1 and/or Table 2. Suitable agents include a plurality of antibodies that bind to a proteins encoded for by a plurality of genes found in Table 1 and/or Table 2.

In other embodiments the invention provides a kit comprising a plurality (e.g. 2-143; 5-135; 10-100; 20-80; 30-60; 40-50) of probes that bind to 2, 3, 4, 5, 6, 8, 10, 12, 14, 16, 18, 20, 30, 40, 50, 60, 70, 80, 90, 100, 110, 120, 130, 140 or more genes found in Table 1 and/or Table 2. In other embodiments the invention provides a kit comprising a plurality (e.g. 2-143; 5-135; 10-100; 20-80; 30-60; 40-50) of antibodies that bind to proteins encoded for by 2, 3, 4, 5, 6, 8, 10, 12, 14, 16, 18, 20, 30, 40, 50, 60, 70, 80, 90, 100, 110, 120, 130, 140 or more the genes found in Table 1 and/or Table 2.

In some embodiments the invention provides a kit for the detection of breast cancer comprising one or more agents that specifically bind one or more markers chosen from the genes listed in Tables 1 and/or Table 2 or FSIP1, CXCL9, MMP7, MMP13 and MMP12. The kit may comprise one or more containers and instructions for determining if the sample is positive for cancer. The kit may optionally contain one or more multiwell plates, a detectable substance such as a dye, a radioactively labeled molecule, a chemiluminescently labeled molecule and the like. The kit may further contain a positive control (e.g. one or more cancerous cells; or specific known quantities of the molecule expressed by the cancer cell) and a negative control (e.g. a tissue or cell sample that is non-cancerous). As an example the kit may take the form of an ELISA or a DNA microarray.

Some embodiments herein are directed to a method of treating breast cancer in a subject, the method comprising administering to a subject in need thereof a therapeutic agent modulating the activity of a cancer associated protein, wherein the cancer associated protein is encoded by gene listed in Table 1 and/or Table 2, homologs thereof, combinations thereof, or a fragment thereof. In some embodiments, the therapeutic agent binds to the breast cancer associated protein. In some embodiments, the therapeutic agent is an antibody. In some embodiments, the antibody may be a monoclonal antibody or a polyclonal antibody. In some embodiments, the antibody is a humanized or human antibody.

Some embodiments herein are directed to a method of treating breast cancer in a subject, the method comprising administering to a subject in need thereof a therapeutic agent modulating the activity of a cancer associated protein, wherein the cancer associated protein is encoded by gene chosen from FSIP1, CXCL9, MMP7, MMP13 and MMP12, homologs thereof, combinations thereof, or a fragment thereof. In some embodiments, the therapeutic agent binds to the breast cancer associated protein. In some embodiments, the therapeutic agent is an antibody. In some embodiments, the antibody may be a monoclonal antibody or a polyclonal antibody. In some embodiments, the antibody is a humanized or human antibody.

In some embodiments, a method of treating breast cancer in a subject may comprise administering to a subject in need thereof a therapeutic agent that modulates the activity of one or more genes chosen from those listed in Table 1 and or Table 2.

In some embodiments, a method of treating breast cancer in a subject may comprise administering to a subject in need thereof a therapeutic agent that modulates the activity of one or more genes chosen from FSIP1, CXCL9, MMP7, MMP13 and MMP12.

In further embodiments, the invention provides a method of treating breast cancer may comprising a gene knockdown of one or more genes listed in Table 1 and/or Table 2. In some embodiments, a method of treating breast cancer may comprise treating cells to knockdown or inhibit expression of a gene encoding an mRNA of one or more genes chosen fromthose listed n Table 1 and or Table 2.

In other embodiments, a method of treating breast cancer may comprise gene knockdown of one or more genes selected from FSIP1, CXCL9, MMP7, MMP13 and MMP12. In some embodiments, a method of treating breast cancer may comprise treating cells to knockdown or inhibit expression of a gene encoding an mRNA of one or more genes chosen from FSIP1, CXCL9, MMP7, MMP13 and MMP12.

In still other embodiments, the present invention provides methods of screening a drug candidate for activity against breast cancer, the method comprising: (a) contacting a cell that expresses one or more cancer associated genes chosen from those listed in Table 1 and/or Table 2 with a drug candidate; (b) detecting an effect of the drug candidate on an expression of the one or more breast cancer associated genes in the cell from a); and (c) comparing the level of expression of one or more of the genes recited in a) in the absence of the drug candidate to the level of expression of the one or more genes in the presence of the drug candidate; wherein a decrease in the expression of the breast cancer associated gene in the presence of the drug candidate indicates that the candidate has activity against breast cancer.

In further embodiments, the present invention provides methods of screening a drug candidate for activity against breast cancer, the method comprising: (a) contacting a cell that expresses one or more breast cancer associated genes chosen from FSIP1, CXCL9, MMP7, MMP 13 and MMP 12 with a drug candidate; (b) detecting an effect of the drug candidate on an expression of the one or more breast cancer associated genes in the cell from a); and (c) comparing the level of expression of one or more of the genes recited in a) in the absence of the drug candidate to the level of expression in the presence of the drug candidate; wherein a decrease in the expression of the breast cancer associated gene in the presence of the drug candidate indicates that the candidate has activity against breast cancer.

In some embodiments, the present invention provides methods of visualizing a breast cancer tumor in a subject comprising a) targeting one or more breast cancer associated proteins with a labeled molecule that binds specifically to the breast cancer tumor, wherein the cancer associated protein is selected from a protein encoded for by one or more genes chosen from those listed in Table 1 and/or Table 2; and b) detecting the labeled molecule, wherein the labeled molecule visualizes the tumor in the subject.

In still other embodiments, the present invention provides methods of visualizing a breast cancer tumor in a subject comprising a) targeting one or more breast cancer associated proteins with a labeled molecule that binds specifically to the breast cancer tumor, wherein the cancer associated protein is selected from a protein encoded for by one or more genes chosen from FSIP1, CXCL9, MMP7, MMP13 and MMP12; and b) detecting the labeled molecule, wherein the labeled molecule visualizes the tumor in the subject.

### BRIEF DESCRIPTION OF THE FIGURES

FIG. 1 shows the serum levels of FSIP1 in breast cancer and normal human subjects.

FIG. 2 shows immunocytochemistry detection of FSIP1 in breast cancer and normal human subjects.

FIG. 3a shows the serum levels of CXCL9 in breast cancer and normal human subjects.

FIG. 3b shows the serum levels of MMP7 in breast cancer and normal human subjects.

FIG. 3c shows the serum levels of MMP12 in breast cancer and normal human subjects.

FIG. 3d shows the serum levels of MMP13 in breast cancer and normal human subjects.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Any methods and materials similar or equivalent to those described herein can be used in the practice or testing of embodiments of the present disclosure.

The invention provides for the rapid, accurate, and cost effective means to detect breast cancer in a subject. The method comprises detecting one or more markers that are specifically expressed on breast cancer tumors in a sample as disclosed infra. The sample may be a bodily fluid such as serum. Thus in some embodiments the invention provides for a non-invasive blood test for detecting breast cancer in a subject.

### DEFINITIONS

As used herein, the singular forms “a,” “an,” and “the” include plural reference unless the context clearly dictates otherwise. Thus, for example, reference to a “therapeutic” is a reference to one or more therapeutics and equivalents thereof known to those skilled in the art, and so forth.

As used herein, the term “about” means plus or minus 5% of the numerical value of the number with which it is being used. Therefore, about 50% means in the range of 45% to 55%.

“Administering,” when used in conjunction with a therapeutic, means to administer a therapeutic directly into or onto a target tissue or to administer a therapeutic to a subject whereby the therapeutic positively impacts the tissue to which it is targeted. Thus, as used herein, the term “administering,” when used in conjunction with a therapeutic, can include, but is not limited to, providing the therapeutic into or onto the target tissue; providing the therapeutic systemically to a subject by, e.g., intravenous injection whereby the therapeutic reaches the target tissue; providing the therapeutic in the form of the encoding sequence thereof to the target tissue (e.g., by so-called gene-therapy techniques). “Administering” a composition may be accomplished by oral administration, intravenous injection, intraperitoneal injection, intramuscular injection, subcutaneous injection, transdermal diffusion or electrophoresis, local injection, extended release delivery devices including locally implanted extended release devices such as bio erodible or reservoir-based implants, as protein therapeutics or as nucleic acid therapeutic via gene therapy vectors, topical administration, or by any of these methods in combination with other known techniques. Such combination techniques include, without limitation, heating, radiation and ultrasound.

The term “subject” as used herein includes, but is not limited to mammals, including humans and non-human primates, farm animals such as pigs, goats, horses, sheep, cows, rodents including rats and mice, rabbits, cats, dogs and the like. In some embodiments, the term “subject,” may refer to humans. In some embodiments, the term “subject,” may refer to a male. In some embodiments, the term “subject,” may refer to a female.

The term “gene product” refers to a nucleotide or protein product that is encoded by a gene. An example of a gene product includes, but is not limited to, mRNA, protein, and the like.

The term “marker” as used herein, includes nucleic acid sequences encoding genes described herein, e.g., genes whose expression level is elevated in breast cancer samples compared to non-cancerous samples, as well as proteins encoded for by those nucleic acid sequneces.

The term “inhibiting” includes the administration of a compound of the present disclosure to prevent the onset of the symptoms, alleviating the symptoms, or eliminating the disease, condition or disorder.

By “pharmaceutically acceptable”, it is meant the carrier, diluent or excipient must be compatible with the other ingredients of the formulation and not deleterious to the recipient thereof.

As used herein, the term “naturally occurring” refers to sequences or structures that may be in a form normally found in nature. “Naturally occurring” may include sequences in a form normally found in any animal.

The term “neoplasm” or “neoplastic condition” refers to an abnormal mass of tissue as a result of neoplasia. Neoplasia can be characterized as an abnormal proliferation of cells. For example, the growth of neoplastic cells exceeds and is not coordinated with that of the normal tissues around it. The growth of a neoplasm can, in some embodiments, persist in the same excessive manner even after cessation of the stimuli. Neoplasms may be, for example, benign, pre-malignant (carcinoma in situ) or malignant (cancer). Accordingly, diagnosing a neoplastic condition as described herein can refer to diagnosing or detecting, for example, benign, pre-malignant, or malignant tumors.

The term “gene expression” refers to a qualitative and/or quantitative result regarding the expression of a gene or gene product. The gene expression result can be an amount or copy number of the gene, or a relative amount of a gene product, the RNA encoded by the gene, the mRNA encoded by the gene, the protein product encoded by the gene, or any combination thereof. The gene expression result can also be normalized or compared to a standard. The gene expression result can be used, for example, to determine if a gene is expressed, overexpressed, or differentially expressed in two or more samples.

The term “capture reagent” refers to a reagent, for example an antibody or antigen binding protein, capable of binding a target molecule or analyte to be detected in a sample.

The terms “specific binding,” “specifically binds,” and the like, refer to instances where two or more molecules form a complex that is measurable under physiologic or assay conditions and is selective. An antibody or antigen binding protein or other molecule is said to “specifically bind” to a protein, antigen, or epitope if, under appropriately selected conditions, such binding is not substantially inhibited, while at the same time non-specific binding is inhibited. Specific binding is characterized by a high affinity and is selective for the compound, protein, epitope, or antigen. Nonspecific binding usually has a low affinity.

Specific binding of IgG antibodies to the epitope specifically recognized by the variable domain of the IgG, for example, is generally characterized by an affinity of at least about 10⁻⁷ M or higher, such as at least about 10⁻⁸ M or higher, or at least about 10⁻⁹ M or higher, or at least about 10⁻¹⁰ or higher, or at least about 10⁻¹¹ M or higher, or at least about 10⁻¹² M or higher. The term is also applicable where, e.g., an antigen-binding domain is specific for a particular epitope that is not carried by numerous antigens, in which case the antibody or antigen binding protein carrying the antigen-binding domain will generally not bind other antigens. In some embodiments, the capture reagent has a Kd equal or less than 10⁻⁹ M, 10⁻¹⁰ M, or 10⁻¹¹ M for its binding partner (e.g. antigen). In some embodiments, the capture reagent has a Ka greater than or equal to 10⁹ M⁻¹ for its binding partner. Capture reagent can also refer to, for example, antibodies.

As used herein, the term “hybridization” or “hybridizing” refers to hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein in reference to nucleic acid molecules refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that a nucleic acid sequence need not be 100% complementary to that of its target nucleic acid to be specifically hybridizable. A nucleic acid compound is specifically hybridizable when there is binding of the molecule to the target, and there is a sufficient degree of complementarity to avoid non-specific binding of the molecule to non-target sequences under conditions in which specific binding is desired, i.e., under physiological conditions in the case of in vivo assays or therapeutic treatment, and in the case of in vitro assays, under conditions in which the assays are performed.

The capture reagents (e.g. antibody) of the present disclosure can also include an anti-antibody, i.e. an antibody that recognizes another antibody but is not specific to an antigen, such as, but not limited to, anti-IgG, anti-IgM, or ant-IgE antibody. This non-specific antibody can be used as a positive control to detect whether the antigen specific antibody is present in a sample.

As used herein, the term “sample” refers to a composition that is being tested or treated with a reagent or analyzed for the presence of cancer. The sample can be used, for example, in a diagnostic test, a screening assay, and the like. In some embodiments, the sample is a biological sample. In some embodiments, the biological sample is blood, plasma, serum, or any combination thereof. A sample can also be derived from blood, plasma, serum, or any combination thereof. Other typical biological samples include, but are not limited to, tissue biopsy, sputum, lymphatic fluid, blood cells (e.g., peripheral blood mononuclear cells), tissue or fine needle biopsy samples, urine, peritoneal fluid, colostrums, breast milk, fetal fluid, fecal material, tears, pleural fluid, or cells therefrom. In some embodiments, the biological sample may comprise urine. A sample can also be derived from any type of biological sample, which means that the sample has been processed in some manner before being used in a method described herein, for example one or more molecules contained within cells found in the sample may be isolated or made accessible to one or more reagents. A sample that has been purified would be considered to be derived from an unpurified sample.

The term “homology,” as used herein, refers to a degree of complementarity. There may be partial homology or complete homology. The word “identity” may substitute for the word “homology.” A partially complementary nucleic acid sequence that at least partially inhibits an identical sequence from hybridizing to a target nucleic acid is referred to as “substantially homologous.” The inhibition of hybridization of the completely complementary nucleic acid sequence to the target sequence may be examined using a hybridization assay (Southern or northern blot, solution hybridization, and the like) under conditions of reduced stringency. A substantially homologous sequence or hybridization probe will compete for and inhibit the binding of a completely homologous sequence to the target sequence under conditions of reduced stringency. This is not to say that conditions of reduced stringency are such that non-specific binding is permitted, as reduced stringency conditions require that the binding of two sequences to one another be a specific (i.e., a selective) interaction. The absence of non-specific binding may be tested by the use of a second target sequence which lacks even a partial degree of complementarity (e.g., less than about 30% homology or identity). In the absence of non-specific binding, the substantially homologous sequence or probe will not hybridize to the second non-complementary target sequence.

The phrases “percent homology,” “% homology,” “percent identity,” or “% identity” refer to the percentage of sequence similarity found in a comparison of two or more amino acid or nucleic acid sequences. Percent identity can be determined electronically, e.g., by using the MEGALIGN program (LASERGENE software package, DNASTAR). The MEGALIGN program can create alignments between two or more sequences according to different methods, e.g., the Clustal Method. (Higgins, D. G. and P. M. Sharp (1988) Gene 73:237-244.) The Clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity. Percent identity between nucleic acid sequences can also be calculated by the Clustal Method, or by other methods known in the art, such as the Jotun Hein Method. (See, e.g., Hein, J. (1990) Methods Enzymol. 183:626-645.) Identity between sequences can also be determined by other methods known in the art, e.g., by varying hybridization conditions.

As used herein, a polynucleotide “derived from” a designated sequence refers to a polynucleotide sequence which is comprised of a sequence of approximately at least about 6 nucleotides, preferably at least about 8 nucleotides, more preferably at least about 10-12 nucleotides, and even more preferably at least about 15-20 nucleotides corresponding to a region of the designated nucleotide sequence. “Corresponding” means homologous to or complementary to the designated sequence. Preferably, the sequence of the region from which the polynucleotide is derived is homologous to or complementary to a sequence that is unique to a cancer associated gene.

In the broadest sense, use of “nucleic acid,” “polynucleotide” or “oligonucleotide” or equivalents herein means at least two nucleotides covalently linked together. In some embodiments, an oligonucleotide is an oligomer of 6, 8, 10, 12, 20, 30 or up to 100 nucleotides. In some embodiments, an oligonucleotide is an oligomer of at least 6, 8, 10, 12, 20, 30, 40, 50, 60, 70, 80, 90, 100, 150, 200, 300, 400, or 500 nucleotides. A nucleic acid, polynucleotide or oligonucleotide may comprise DNA, RNA, PNA or a polymer of nucleotides linked by phosphodiester and/or any alternate bonds.

Similarly, a “recombinant protein” is a protein made using recombinant techniques, for example, but not limited to, through the expression of a recombinant nucleic acid as depicted above. A recombinant protein may be distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises about 50-75%, about 80%, or about 90%. In some embodiments, a substantially pure protein comprises about 80-99%, 85-99%, 90-99%, 95-99%, or 97-99% by weight of the total protein. A recombinant protein can also include the production of a cancer associated protein from one organism (e.g. human) in a different organism (e.g. yeast, E. coli, or the like) or host cell. Alternatively, the protein may be made at a significantly higher concentration than is normally seen, through the use of an inducible promoter or high expression promoter, such that the protein is made at increased concentration levels. Alternatively, the protein may be in a foiin not normally found in nature, as in the addition of an epitope tag or amino acid substitutions, insertions and deletions, as discussed herein.

As used herein, the term “tag,” “sequence tag” or “primer tag sequence” refers to an oligonucleotide with specific nucleic acid sequence that serves to identify a batch of polynucleotides bearing such tags therein. Polynucleotides from the same biological source are covalently tagged with a specific sequence tag so that in subsequent analysis the polynucleotide can be identified according to its source of origin. The sequence tags also serve as primers for nucleic acid amplification reactions.

A “microarray” is a linear or two-dimensional array of, for example, discrete regions, each having a defined area, formed on the surface of a solid support. The density of the discrete regions on a microarray is determined by the total numbers of target polynucleotides to be detected on the surface of a single solid phase support, preferably at least about 50/cm², more preferably at least about 100/cm², even more preferably at least about 500/cm², and still more preferably at least about 1,000/cm². As used herein, a DNA microarray is an array of oligonucleotide primers placed on a chip or other surfaces used to identify, amplify, detect, or clone target polynucleotides. Since the position of each particular group of primers in the array is known, the identities of the target polynucleotides can be determined based on their binding to a particular position in the microarray.

The term “label” or “detectable substance” refers to a composition capable of producing a detectable signal indicative of the presence of the target polynucleotide, protein or peptide in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by a device or method, such as, but not limited to, a spectroscopic, photochemical, biochemical, immunochemical, electrical, optical, chemical detection device or any other appropriate device. In some embodiments, the label may be detectable visually without the aid of a device. The Willi “label” is used to refer to any chemical group or moiety having a detectable physical property or any compound capable of causing a chemical group or moiety to exhibit a detectable physical property, such as an enzyme that catalyzes conversion of a substrate into a detectable product. The term “label” also encompasses compounds that inhibit the expression of a particular physical property. The label may also be a compound that is a member of a binding pair, the other member of which bears a detectable physical property.

The term “support” refers to conventional supports such as beads, particles, dipsticks, fibers, filters, membranes, and silane or silicate supports such as glass slides.

The term “amplify” is used in the broad sense to mean creating an amplification product which may include, for example, additional target molecules, or target-like molecules or molecules complementary to the target molecule, which molecules are created by virtue of the presence of the target molecule in the sample. In the situation where the target is a nucleic acid, an amplification product can be made enzymatically with DNA or RNA polymerases or reverse transcriptases, or any combination thereof.

The term “biological sources” as used herein refers to the sources from which the target polynucleotides may be derived. The source can be of any form of “sample” as described above, including but not limited to, cell, tissue or fluid. “Different biological sources” can refer to different cells/tissues/organs of the same individual, or cells/tissues/organs from different individuals of the same species, or cells/tissues/organs from different species.

As used herein, the term “therapeutic” or “therapeutic agent” means an agent that can be used to treat, combat, ameliorate, prevent or improve an unwanted condition or disease of a patient. In part, embodiments of the present disclosure are directed to the treatment of cancer, neoplasm, or the decrease in proliferation of cells. In some embodiments, the term “therapeutic” or “therapeutic agent” may refer to any molecule that associates with or affects the target marker, its expression or its function. In various embodiments, such therapeutics may include molecules such as, for example, a therapeutic cell, a therapeutic peptide, a therapeutic gene, a therapeutic compound, or the like, that associates with or affects the target marker, its expression or its function.

A “therapeutically effective amount” or “effective amount” of a composition is a predetermined amount calculated to achieve the desired effect, i.e., to inhibit, block, or reverse the activation, migration, or proliferation of cells. In some embodiments, the effective amount is a prophylactic amount. In some embodiments, the effective amount is an amount used to treat the disease or condition. The specific dose of a composition administered according to this invention to obtain therapeutic and/or prophylactic effects will, of course, be determined by the particular circumstances surrounding the case, including, for example, the composition administered, the route of administration, and the condition being treated. It will be understood that the effective amount administered will be determined by the physician in the light of the relevant circumstances including the condition to be treated, the choice of composition to be administered, and the chosen route of administration. A therapeutically effective amount of composition of this invention is typically an amount such that when it is administered in a physiologically tolerable excipient composition, it is sufficient to achieve an effective systemic concentration or local concentration in the targeted tissue.

The terms “treat,” “treated,” or “treating” as used herein can refer to both therapeutic treatment or prophylactic or preventative measures, wherein the object is to prevent or slow down (lessen) an undesired physiological condition, disorder or disease, or to obtain beneficial or desired clinical results. In some embodiments, the term may refer to both treating and preventing. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. Treat, treated, or treating may include inhbiting the growth a breast cancer tumor and/or inhibiting the metastasis of a breast cancer tumor.

Generally speaking, the term “tissue” refers to any aggregation of similarly specialized cells that are united in the performance of a particular function.

### Breast Cancer Associated Molecules

In some embodiments, the present disclosure provides for nucleic acid and protein sequences that are associated with breast cancer, herein termed “cancer associated” or “CA” sequences found in a subject. In some embodiments, the term “cancer associated sequences” may indicate that the nucleotide or protein sequences are differentially expressed, activated, inactivated or altered in cancers as compared to normal tissue. Cancer associated sequences may include those that are up-regulated (i.e. expressed at a higher level), as well as those that are down-regulated (i.e. expressed at a lower level), in cancers. Cancer associated sequences can also include sequences that have been altered (i.e., translocations, truncated sequences or sequences with substitutions, deletions or insertions, including, but not limited to, point mutations) and show either the same expression profile or an altered profile. Also included are splice variants and the like. Cancer associated sequences, such as cancer associated proteins, can also be associated with neoplasms.

Suitable examples of breast cancer markers include nucleic acids and/or proteins encoded for by genes including FSIP1 (Accession No. NM_{—}152597.4), CXCL9 (Accession No. NM_{—}002416.1), MMPI (Accession No. NM_{—}002423.3), MMP13 (Accession No. NM_{—}002427.2) and MMP12 (Accession No. NM_{—}002426.2). Other suitable examples of breast cancer markers include the genes listed in Tables 1 and 2.

In some embodiments, cancer associated sequences may include both nucleic acid and amino acid sequences. In some embodiments, the cancer associated sequences may include sequences having at least about 60% homology with the disclosed sequences. In some embodiments, the cancer associated sequences may have at least about 65%, about 70%, about 75%, about 80%, about 85%, about 90%, about 95%, about 97%, about 99%, about 99.8% homology with the disclosed sequences. In some embodiments, the cancer associated sequences may be “mutant nucleic acids”. As used herein, “mutant nucleic acids” refers to deletion mutants, insertions, point mutations, substitutions, translocations.

A nucleic acid of the present disclosure may include phosphodiester bonds, although in some cases, as outlined below (for example, in antisense applications or when a nucleic acid is a candidate drug agent), nucleic acid analogs may have alternate backbones, comprising, for example, phosphoramidate (Beaucage et al., Tetrahedron 49(10):1925 (1993) and references therein; Letsinger, J. Org. Chem. 35:3800 (1970); Sprinzl et al., Eur, J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem, Int. Ed. Engl. 31:1008 (1992); Nielsen, Nature, 365:566 (1993); Carlsson et al., Nature 380:207 (1996). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp 169-176). Several nucleic acid analogs are described in Rawls, C & E News Jun. 2, 1997 page 35. These modifications of the ribose-phosphate backbone may be done for a variety of reasons, for example to increase the stability and half-life of such molecules in physiological environments for use in anti-sense applications or as probes on a biochip.

As will be appreciated by those skilled in the art, such nucleic acid analogs may be used in some embodiments of the present disclosure. In addition, mixtures of naturally occurring nucleic acids and analogs can be made; alternatively, mixtures of different nucleic acid analogs, and mixtures of naturally occurring nucleic acids and analogs may be made.

In some embodiments, the nucleic acids may be single stranded or double stranded or may contain portions of both double stranded or single stranded sequence. As will be appreciated by those skilled in the art, the depiction of a single strand also defines the sequence of the other strand; thus the sequences described herein also includes the complement of the sequence. The nucleic acid may be DNA, both genomic and cDNA, RNA, or a hybrid, where the nucleic acid contains any combination of deoxyribo- and ribo-nucleotides, and any combination of bases, including uracil, adenine, thymine, cytosine, guanine, inosine, xanthine, hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus, for example, the subject units of a peptide nucleic acid, each containing a base, are referred to herein as a nucleoside.

In some embodiments, the cancer associated sequences may be recombinant nucleic acids. By the term “recombinant nucleic acid” herein refers to nucleic acid molecules, originally formed in vitro, in general, by the manipulation of nucleic acid by polymerases and endonucleases, in a form not normally found in nature. Thus a recombinant nucleic acid may also be an isolated nucleic acid, in a linear form, or cloned in a vector fowled in vitro by ligating DNA molecules that are not normally joined, are both considered recombinant for the purposes of this invention. It is understood that once a recombinant nucleic acid is made and reintroduced into a host cell or organism, it can replicate using the in vivo cellular machinery of the host cell rather than in vitro manipulations; however, such nucleic acids, once produced recombinantly, although subsequently replicated in vivo, are still considered recombinant or isolated for the purposes of the invention. As used herein, a “polynucleotide” or “nucleic acid” is a polymeric foHn of nucleotides of any length, either ribonucleotides or deoxyribonucleotides. This term includes double- and single-stranded DNA and RNA. It also includes known types of modifications, for example, labels which are known in the art, methylation, “caps”, substitution of one or more of the naturally occurring nucleotides with an analog, internucleotide modifications-such as, for example, those with uncharged linkages (e.g., phosphorothioates, phosphorodithioates, etc.), those containing pendant moieties, such as, for example proteins (including e.g., nucleases, toxins, antibodies, signal peptides, poly-L-lysine, etc.), those with intercalators (e.g., acridine, psoralen, etc.), those containing chelators (e.g., metals, radioactive metals, etc.), those containing alkylators, those with modified linkages (e.g., alpha anomeric nucleic acids, etc.), as well as unmodified forms of the polynucleotide.

### Breast Cancer Detection

In some embodiments the invention provides for the rapid, specific, sensitive, non-invasive and cost effective method of detecting breast cancer in a subject. The method comprises detecting one or more markers disclosed infra as being associated with breast cancer in a subject. The method may include comparing the expression level of a marker in a sample, such as one obtained from a subject, with the expression level in normal breast tissue. Expression levels above that found within normal tissue may indicate the presence of breast cancer in the subject. The method may include comparing the level of detection of the marker with a sample obtained from a subject having breast cancer. A level of expression in the sample similar to (e.g. within 10% of the level found in the breast cancer sample) or higher than that found in the breast cancer sample may indicate the presence of breast cancer in the subject.

The detection of the expression level of the one or more markers disclosed infra may be by any means known in the art. For example where the marker is a protein associated with breast cancer an ELISA may used to detect the expression level of the marker. Other suitable assays for detecting the presence of a protein marker include a radio-immunoassay, a western blot, an immunoprecipitation assay, such as a bead based assay, e.g a magnetic bead based assay. In some embodiments the marker may be isolated from the sample before detection, but in other embodiments it is not isolated from the sample. In some embodiments the protein marker may be expressed in a cellular context (i.e. on the surface of the cell or within the cell). In these instances immunocytochemistry may be used to detect the marker. Alternatively, the flow cytometry can used to detect the marker. Where the marker is contained within the cell, the cells may be treated with a detergent to make the marker accessible to a detection reagent. Suitable detection reagents would include any molecule that specifically binds the marker, such as an antibody that specifically binds to an epitope on the marker.

Suitable agents for detecting a protein marker as disclosed infra include any specific binding partner of the breast cancer marker. For example the specific binding partner may be a protein that binds the breast cancer marker, such as an antibody. Other suitable specific binding partners may include a receptor that binds the breast cancer marker or an enzyme that specifically binds the breast cancer marker.

The cancer can also be diagnosed to a specific tissue type as well by visualizing the labeled molecule. The molecule can be visualized or detected using any method, such as but not limited to, MRI, CAT scan, PET scan, and the like. In some embodiments, an antibody can bind to the protein and then be detected. In some embodiments, the level of antibody binding can be quantified to determine whether the protein is overexpressed. Differential expression can also be determined by known methods. Accordingly, embodiments hereof provide a method for imaging structures in tissues and cells of a subject having cancer, is suspected of having cancer, or is undergoing a diagnostic procedure to determine if the person has cancer. If the imaging demonstrates that the cancer associated protein is overexpressed or differentially expressed then the patient is diagnosed as having cancer or suspected of having cancer. Other tests can also be done, such as but not limited to, a biopsy to confirm, or otherwise aid, the diagnosis.

The label molecules can also be labeled by, but not limited to, any radioisotopes that can be imaged with a PET or SPECT camera. For example, radiopharmaceuticals of various embodiments may be radiolabeled with radioisotopes such as, but not limited to, ⁷⁶Br, ¹²³I, ¹²⁵I, ¹³¹I, ^{99m}Tc, ¹¹C, ¹⁸F, or other gamma- or positron-emitting radionuclides. In other embodiments, the label molecules may be radiolabeled with a combination of radioisotopes.

In some embodiments the marker associated with breast cancer may be a nucleic acid, e.g. an mRNA molecule. The nucleic acid may be isolated from the sample. Detection of the nucleic acid may be by any means known in the art. For example the nucleic acid molecule may be detected by Southern blot or northern blot mass spectroscopy, microarray and the like. The nucleic acid may be detected using PCR, for example where the nucleic acid is an RNA molecule, such as an mRNA molecule, rtPCR may be used. The PCR may be quantitative PCR (e.g. qPCT) or real time PCR. The nucleic acid may be detected by in situ hybridization where the sample includes breast cancer cells.

The assays described above may include the use of a probe to detect the nucleic acid marker. Probes are described infra. Briefly, the probe may be a nucleic acid molecule ranging from 5-40, 10-35, 15-30 nucleotides long. The probe may be about 5, about 10, about 20, about 25, about 30, about 35 nucleotides long. The probe may include a portion of a gene encoding the breast cancer marker, or a complement of a gene encoding a breast cancer marker. Examples of suitable probes are provided in Tables 1 and 2.

The gene expression levels may be represented as relative expression normalized to the ADPRT (Accession number NM_{—}001618.2,), GAPD (Accession number NM_{—}002046.2,), or other housekeeping genes known in the art. In the case of microarrayed probes of mRNA expression, the gene expression data may also be normalized by a median of medians method. In this method, each array gives a different total intensity. Using the median value is a robust way of comparing cell lines (arrays) in an experiment. As an example, the median was found for each cell line and then the median of those medians became the value for normalization. The signal from the each cell line was made relative to each of the other cell lines.

RNA extraction: Cells of the present disclosure may be incubated with 0.05% trypsin and 0.5 mM EDTA, followed by collecting in DMEM (Gibco, Gaithersburg, Md.) with 0.5% BSA. Total RNA may be purified from cells using the RNeasy Mini kit (Qiagen, Germany).

The use of microarray analysis of gene expression allows the identification of host sequences associated with breast cancer or neoplasia. These sequences may then be used in a number of different ways, including diagnosis, prognosis, screening for modulators (including both agonists and antagonists), antibody generation (for immunotherapy and imaging), etc.

In addition to a comparison between normal and a non-normal sample, overexpression can be determined based upon a cutoff value. Examples of non-limiting cutoff values are described herein. Overexpression can be determined, for example, at the genetic level, nucleotide expression level (e.g. mRNA expression), or protein level (e.g. concentration of the protein product encoded by a gene in a sample, such as but not limited to blood, urine, or serum).

In some embodiments, the invention provides a method for detecting cancer by detecting the presence of an antibody in a test serum sample. In some embodiments, the antibody recognizes a polypeptide or an epitope thereof disclosed herein. In some embodiments, the antibody recognizes a polypeptide or epitope thereof encoded by a nucleic acid sequence disclosed herein. In some embodiments, the method comprises detecting a level of an antibody against an antigenic polypeptide such as, without limitation, a cancer associated protein, or an antigenic fragment thereof. In some embodiments, the method comprises comparing the level of the antibody in the test sample with a level of the antibody in the control sample, wherein an altered level of antibody in said test sample relative to the level of antibody in the control sample is indicative of the presence of cancer in the test sample. In some embodiments, the control sample is a sample derived from a normal cell or non-cancerous sample. In some embodiments, the control is derived from a cancer sample, and, therefore, in some embodiments, the method comprises comparing the levels of binding and/or the amount of antibody in the sample, wherein when the levels or amount are the same as the cancer control sample is indicative of the presence of cancer in the test sample.

### Methods of Treating Breast Cancer

In other embodiments, a method of treating breast cancer may comprise administering an antibody against the protein to a subject in need thereof. In some embodiments, the antibody may be a monoclonal antibody or a polyclonal antibody. In some embodiments, the antibody may be a humanized or a recombinant antibody. Antibodies can be made that specifically bind to this region using known methods and any method is suitable. In some embodiments, the antibody specifically binds to a sequence encoded by a fragment thereof, a complement thereof or a combination thereof.

In some embodiments, cancers or neoplasia expressing one of the cancer associated sequences may be treated by antagonizing the cancer associated sequence's activity. In some embodiments, a method of treating cancer or neoplasia may comprise administering a therapeutic such as, without limitation, antibodies that antagonize the ligand binding to the cancer associated sequence, small molecules that inhibit the cancer associated sequence's expression or activity, siRNAs directed towards the cancer associated sequence, or the like.

In some embodiments, a method of treating cancer comprises detecting the presence of a cancer associated sequence's receptor and administering a cancer treatment. The cancer treatment may be any cancer treatment or one that specifically inhibits the action of a cancer associated sequence or receptor. In some embodiments, therefore, a sample would be obtained from the subject and tested for the presence of a cancer associated sequence or the overexpression of a cancer associated sequence as described herein. In some embodiments, if a cancer associated sequence described herein is found to be overexpressed a cancer treatment or therapeutic is administered to the subject. The cancer treatment may be a conventional non-specific treatment, such as chemotherapy, or the treatment may comprise a specific treatment that only targets the activity of the cancer associated sequence or the receptor to which the cancer associated sequence binds. These treatments can be, for example, an antibody that specifically binds to the cancer associated sequence and inhibits its activity.

In some embodiments, a method of identifying an anti-cancer agent is provided, wherein the method comprises contacting a candidate agent to a sample; and determining the cancer associated sequence's activity in the sample. In some embodiments, the candidate agent is identified as an anti-cancer agent if the cancer associated sequence's activity is reduced in the sample after the contacting. In some embodiments, the candidate agent is a candidate antibody. In some embodiments, the method comprises contacting a candidate antibody that binds to the cancer associated sequence with a sample, and assaying for the cancer associated sequence's activity, wherein the candidate antibody is identified as an anti-cancer agent if the cancer associated sequence activity is reduced in the sample after the contacting. A cancer associated sequence's activity can be any activity of the cancer associated sequence.

In some embodiments, a method of treating breast cancer may comprise administering an agent that interferes with the synthesis, secretion, receptor binding or receptor signaling of cancer associated proteins or its receptors.

In some embodiments, a method of treating cancer may comprise gene knockdown of one or more cancer associated sequences described herein. Gene knockdown refers to techniques by which the expression of one or more of an organism's genes is reduced, either through genetic modification (a change in the DNA of one of the organism's chromosomes such as, without limitation, chromosomes encoding cancer associated sequences) or by treatment with a reagent such as a short DNA or RNA oligonucleotide with a sequence complementary to either an mRNA transcript or a gene. In some embodiments, the oligonucleotide used may be selected from RNase-H competent antisense, such as, without limitation, ssDNA oligonucleotides, ssRNA oligonucleotides, phosphorothioate oligonucleotides, or chimeric oligonucleotides; RNase-independent antisense, such as morpholino oligonucleotides, 2′-O-methyl phosphorothioate oligonucleotides, locked nucleic acid oligonucleotides, or peptide nucleic acid oligonucleotides; RNAi oligonucleotides, such as, without limitation, siRNA duplex oligonucleotides, or shRNA oligonucleotides; or any combination thereof. In some embodiments, a plasmid may be introduced into a cell, wherein the plasmid expresses either an antisense RNA transcript or an shRNA transcript. The oligo introduced or transcript expressed may interact with the target mRNA (ex. a sequence disclosed in Table 1) by complementary base pairing (a sense-antisense interaction).

The specific mechanism of silencing may vary with the oligo chemistry. In some embodiments, the binding of a oligonucleotide described herein to the active gene or its transcripts may cause decreased expression through blocking of transcription, degradation of the mRNA transcript (e.g. by small interfering RNA (siRNA) or RNase-H dependent antisense) or blocking either mRNA translation, pre-mRNA splicing sites or nuclease cleavage sites used for maturation of other functional RNAs such as miRNA (e.g. by Morpholino oligonucleotides or other RNase-H independent antisense). For example, RNase-H competent antisense oligonucleotides (and antisense RNA transcripts) may form duplexes with RNA that are recognized by the enzyme RNase-H, which cleaves the RNA strand. As another example, RNase-independent oligonucleotides may bind to the mRNA and block the translation process. In some embodiments, the oligonucleotides may bind in the 5′-UTR and halt the initiation complex as it travels from the 5′-cap to the start codon, preventing ribosome assembly. A single strand of RNAi oligonucleotides may be loaded into the RISC complex, which catalytically cleaves complementary sequences and inhibits translation of some mRNAs bearing partially-complementary sequences. The oligonucleotides may be introduced into a cell by any technique including, without limitation, electroporation, microinjection, salt-shock methods such as, for example, CaCl2 shock; transfection of anionic oligo by cationic lipids such as, for example, Lipofectamine; transfection of uncharged oligonucleotides by endosomal release agents such as, for example, Endo-Porter; or any combination thereof. In some embodiments, the oligonucleotides may be delivered from the blood to the cytosol using techniques selected from nanoparticle complexes, virally-mediated transfection, oligonucleotides linked to octaguanidinium dendrimers (Morpholino oligonucleotides), or any combination thereof.

### Eliciting an Immune Response Against a Breast Cancer Antigen

In some embodiments a cancer antigen as described herein may be used to elicit an immune response from a subject. For example at least a portion of a protein encoded for by a breast cancer gene may be injected into a subject to elicit an immune response, such as an antibody response. The antibody may be isolated from the subject and used as a research, dsiagnostic or thereapeutic reagent.

In some embodiments, a cancer associated expression sequence may be introduced (e.g., transduced) into DCs or stem cells in any of a variety of standard methods, including transfection, recombinant vaccinia viruses, adeno-associated viruses (AAVs), retroviruses, etc.

In some embodiments, the transformed DCs of the invention may be introduced into the subject (e.g., without limitation, a human patient) where the DCs may induce an immune response. Typically, the immune response includes a cytotoxic T-lymphocyte (CTL) response against target cells bearing antigenic peptides (e.g., in a MHC class I/peptide complex). These target cells are typically cancer cells.

In some embodiments, when the DCs are to be administered to a subject, they may preferably isolated from, or derived from precursor cells from, that subject (i.e., the DCs may administered to an autologous subject). However, the cells may be infused into HLA-matched allogeneic or HLA-mismatched allogeneic subject. In the latter case, immunosuppressive drugs may be administered to the subject.

In some embodiments, the cells may be administered in any suitable manner. In some embodiments, the cell may be administered with a pharmaceutically acceptable carrier (e.g., saline). In some embodiments, the cells may be administered through intravenous, intra-articular, intramuscular, intradermal, intraperitoneal, or subcutaneous routes. Administration (i.e., immunization) may be repeated at time intervals. Infusions of DC may be combined with administration of cytokines that act to maintain DC number and activity (e.g., GM-CSF, IL-12).

In some embodiments, the dose administered to a subject may be a dose sufficient to induce an immune response as detected by assays which measure T cell proliferation, T lymphocyte cytotoxicity, and/or effect a beneficial therapeutic response in the subject over time, e.g., to inhibit growth of cancer cells or result in reduction in the number of cancer cells or the size of a tumor.

In some embodiments, DCs are obtained (either from a patient or by in vitro differentiation of precursor cells) and pulsed with antigenic peptides having a cancer associated sequence. The pulsing results in the presentation of peptides onto the surface MHC molecules of the cells. The peptide/MHC complexes displayed on the cell surface may be capable of inducing a MHC-restricted cytotoxic T-lymphocyte response against target cells expressing cancer associated polypeptides (e.g., without limitations, cancer cells).

In some embodiments, cancer associated sequences used for pulsing may have at least about 6 or 8 amino acids and fewer than about 30 amino acids or fewer than about 50 amino acid residues in length. In some embodiments, an immunogenic peptide sequence may have from about 8 to about 12 amino acids. In some embodiments, a mixture of human protein fragments may be used; alternatively a particular peptide of defined sequence may be used. The peptide antigens may be produced by de novo peptide synthesis, enzymatic digestion of purified or recombinant human peptides, by purification of the peptide sequence from a natural source (e.g., a subject or tumor cells from a subject), or expression of a recombinant polynucleotide encoding a human peptide fragment.

In some embodiments, the amount of peptide used for pulsing DC may depend on the nature, size and purity of the peptide or polypeptide. In some embodiments, an amount of from about 0.05 ug/ml to about 1 mg/ml, from about 0.05 ug/ml to about 500 ug/ml, from about 0.05 ug/ml to about 250 ug/ml, from about 0.5 ug/ml to about 1 mg/ml, from about 0.5 ug/ml to about 500 ug/ml, from about 0.5 ug/ml to about 250 ug/ml, or from about 1 ug/ml to about 100 ug/ml of peptide may be used. After adding the peptide antigen(s) to the cultured DC, the cells may then be allowed sufficient time to take up and process the antigen and express antigen peptides on the cell surface in association with either class I or class II MHC. In some embodiments, the time to take up and process the antigen may be about 18 to about 30 hours, about 20 to about 30 hours, or about 24 hours.

It will be appreciated that there are various methods of obtaining expression data and uses of the expression data. For example, the expression data that can be used to detect or diagnose a subject with cancer can be obtained experimentally. In some embodiments, obtaining the expression data comprises obtaining the sample and processing the sample to experimentally determine the expression data. The expression data can comprise expression data for one or more of the cancer associated sequences described herein. The expression data can be experimentally determined by, for example, using a microuray or quantitative amplification method such as, but not limited to, those described herein. In some embodiments, obtaining expression data associated with a sample comprises receiving the expression data from a third party that has processed the sample to experimentally determine the expression data.

### Breast Cancer Drug Screening

In some embodiments, a method of screening drug candidates includes comparing the level of expression of the cancer-associated sequence in the absence of the drug candidate to the level of expression in the presence of the drug candidate.

Some embodiments are directed to a method of screening for a therapeutic agent capable of binding to a cancer-associated sequence (nucleic acid or protein), the method comprising combining the cancer-associated sequence and a candidate therapeutic agent, and determining the binding of the candidate agent to the cancer-associated sequence.

Further provided herein is a method for screening for a therapeutic agent capable of modulating the activity of a cancer-associated sequence. In some embodiments, the method comprises combining the cancer-associated sequence and a candidate therapeutic agent, and determining the effect of the candidate agent on the bioactivity of the cancer-associated sequence. An agent that modulates the bioactivity of a cancer associated sequence may be used as a therapeutic agent capable of modulating the activity of a cancer-associated sequence.

In some embodiments, a method of evaluating the effect of a candidate cancer drug may comprise administering the drug to a subject and removing a cell sample from the subject. The expression profile of the cell is then detet ined. In some embodiments, the method may further comprise comparing the expression profile of the subject to an expression profile of a healthy individual. In some embodiments, the expression profile comprises measuring the expression of one or more or any combination of the sequences disclosed herein. In some embodiments, where the expression profile of one or more or any combination of the sequences disclosed herein is modified (e.g., increased or decreased), the candidate cancer drug is said to be effective.

In some embodiments, the method comprises detecting an effect of the cancer drug candidate on an expression of the cancer associated polynucleotide in the cell or an effect on the cell's growth or viability. In some embodiments, the method comprises comparing the level of expression, cell growth, or viability in the absence of the drug candidate to the level of expression, cell growth, or viability in the presence of the drug candidate; wherein an effect on the expression of the cancer associated polynucleotide, cell growth, or viability indicates that the candidate has activity against a cancer cell that overexpresses a cancer associated gene, wherein said gene comprises a sequence that is a sequence selected from the sequences, genes, or gene products disclosed herein or complementary thereto, homologs thereof, combinations thereof, or fragments thereof. In some embodiments, the drug candidate is selected from a transcription inhibitor, a G-protein coupled receptor antagonist, a growth factor antagonist, a serine-threonine kinase antagonist, or a tyrosine kinase antagonist.

### Screening Platforms

Some embodiments are directed to a biochip comprising a nucleic acid segment which encodes a cancer associated protein. In some embodiments, a biochip comprises a nucleic acid molecule which encodes at least a portion of a cancer associated protein. In some embodiments, the cancer associated protein is encoded by a sequence selected from a sequence, gene or gene product disclosed herein, homologs thereof, combinations thereof, or a fragment thereof. In some embodiments, the nucleic acid molecule specifically hybridizes with a nucleic acid sequence selected from a sequence or gene described herein. Examples of genes include, but are not limited to, any of the genes listed in Table 1, FSIP1, CXCL9, MMP7, MMP13 and MMP12, a homolog thereof, a fragment thereof, a complement thereof or a combination thereof.

In some embodiments, the biochip comprises a first and second nucleic molecule wherein the first nucleic acid molecule specifically hybridizes with a first sequence selected from a sequence that encodes for, or is encoded by genes listed in Table 1, FSIP1, CXCL9, MMP7, MMP13 and MMP12 a homolog thereof, a fragment thereof, a complement thereof or a combination thereof and the second nucleic acid molecule specifically hybridizes with a second sequence selected from a sequence or gene selected from genes listed in Table 1, FSIP1, CXCL9, MMP7, MMP13 and MMP12 a homolog thereof, a fragment thereof, a complement thereof or a combination thereof or, wherein the first and second sequences are not the same sequence. In some embodiments, the present invention provides methods of detecting or diagnosing cancer comprising detecting the expression of a nucleic acid sequence selected from a sequence or gene disclosed herein, wherein a sample is contacted with a biochip comprising a sequence selected from a sequence disclosed or sequence encoded by a gene disclosed herein, homologs thereof, combinations thereof, or a fragment thereof.

In some embodiments, an isolated nucleic acid comprises at least 10, 12, 15, 20 or 30 contiguous nucleotides of a sequence selected from the group consisting of the cancer associated polynucleotide sequences encoding for, or encoded by genes listed in Table 1, FSIP1, CXCL9, MMP7, MMP13 and MMP12 a homolog thereof, a fragment thereof, a complement thereof or a combination thereof.

In some embodiments, the polynucleotide, or its complement or a fragment thereof, further comprises a detectable label, is attached to a solid support, is prepared at least in part by chemical synthesis, is an antisense fragment, is single stranded, is double stranded or comprises a microarray.

In some embodiments, the present invention provides an isolated polypeptide, encoded within an open reading frame of a cancer associated sequence selected from the polynucleotide sequences encoded by a gene described herein, or its complement. In some embodiments, the invention provides an isolated polypeptide, wherein said polypeptide comprises the amino acid sequence encoded by a polynucleotide selected from the group consisting of the sequences encoding for genes listed in Table 1, FSIP1, CXCL9, MMPI, MMP 13 and MMP 12, a homolog thereof, a fragment thereof, a complement thereof or a combination thereof. In some embodiments, the invention provides an isolated polypeptide, wherein said polypeptide comprises the amino acid sequence encoded by a cancer associated polypeptide.

### Pharmaceutical Formulations and Administration of Therapeutics

Modes of administration for a therapeutic (either alone or in combination with other pharmaceuticals) can be, but are not limited to, sublingual, injectable (including short-acting, depot, implant and pellet farms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication. The selection of the specific route of administration and the dose regimen is to be adjusted or titrated by the clinician according to methods known to the clinician in order to obtain the optimal clinical response. The amount of therapeutic to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

Pharmaceutical formulations containing the therapeutic of the present disclosure and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present disclosure. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compositions of the present disclosure can be formulated for parenteral administration by injection, e.g., by bolus injection or continuous infusion. The compositions can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compositions can be formulated readily by combining the therapeutic with pharmaceutically acceptable carriers well known in the art. Such carriers enable the therapeutic of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active therapeutic doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active therapeutic can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the pharmaceutical compositions can take the form of, e.g., tablets or lozenges formulated in a conventional manner.

For administration by inhalation, the therapeutic for use according to the present disclosure is conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the therapeutic and a suitable powder base such as lactose or starch.

The compositions of the present disclosure can also be formulated in rectal compositions such as suppositories or retention enemas, e.g., containing conventional suppository bases such as cocoa butter or other glycerides.

In addition to the formulations described previously, the therapeutic of the present disclosure can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compositions can be formulated with suitable polymeric or hydrophobic materials (for example as an emulsion in an acceptable oil) or ion exchange resins, or as sparingly soluble derivatives, for example, as a sparingly soluble salt.

In transdermal administration, the compositions of the present disclosure, for example, can be applied to a plaster, or can be applied by transdermal, therapeutic systems that are consequently supplied to the organism.

Pharmaceutical compositions can include suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compositions of the present disclosure can also be administered in combination with other active ingredients, such as, for example, adjuvants, protease inhibitors, or other compatible drugs or compounds where such combination is seen to be desirable or advantageous in achieving the desired effects of the methods described herein.

In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments, the diluent component may include one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the optional lubricant component, when present, comprises one or more of stearic acid, metallic stearate, sodium stearylfumarate, fatty acid, fatty alcohol, fatty acid ester, glycerylbehenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

### Kits

Also provided by the subject invention are kits and systems for practicing the subject methods, as described above, such components configured to diagnose cancer in a subject, treat cancer in a subject, or perform basic research experiments on cancer cells (e.g., either derived directly from a subject, grown in vitro or ex vivo, or from an animal model of cancer. The various components of the kits may be present in separate containers or certain compatible components may be pre-combined into a single container, as desired.

The subject systems and kits may also include one or more other reagents for performing any of the subject methods. The reagents may include one or more matrices, solvents, sample preparation reagents, buffers, desalting reagents, enzymatic reagents, denaturing reagents, probes, polynucleotides, vectors (e.g., plasmid or viral vectors), etc., where calibration standards such as positive and negative controls may be provided as well. As such, the kits may include one or more containers such as vials or bottles, with each container containing a separate component for carrying out a sample processing or preparing step and/or for carrying out one or more steps for producing a normalized sample according to the present disclosure.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the subject database, programming and instructions, the kits may also include one or more control samples and reagents, e.g., two or more control samples for use in testing the kit.

### Antibodies for Use in the Invention

Intact antibodies, also known as immunoglobulins, are typically tetrameric glycosylated proteins composed of two light (L) chains of approximately 25 kDa each, and two heavy (H) chains of approximately 50 kDa each. Two types of light chain, termed lambda and kappa, exist in antibodies. Depending on the amino acid sequence of the constant domain of heavy chains, immunoglobulins are assigned to five major classes: A, D, E, G, and M, and several of these may be further divided into subclasses (isotypes), e.g., IgG1, IgG2, IgG3, IgG4, IgA1, and IgA2. Each light chain is composed of an N-terminal variable (V) domain (VL) and a constant (C) domain (CL). Each heavy chain is composed of an N-terminal V domain (VH), three or four C domains (CHs), and a hinge region. The CH domain most proximal to VH is designated CHI. The VH and VL domains consist of four regions of relatively conserved sequences named framework regions (FR1, FR2, FR3, and FR4), which form a scaffold for three regions of hypervariable sequences (complementarity determining regions, CDRs). The CDRs contain most of the residues responsible for specific interactions of the antibody or antigen binding protein with the antigen. CDRs are referred to as CDR1, CDR2, and CDR3. Accordingly, CDR constituents on the heavy chain are referred to as H1, H2, and H3, while CDR constituents on the light chain are referred to as L1, L2, and L3. CDR3 is the greatest source of molecular diversity within the antibody or antigen binding protein-binding site. H3, for example, can be as short as two amino acid residues or greater than 26 amino acids. The subunit structures and three-dimensional configurations of different classes of immunoglobulins are well known in the art. For a review of the antibody structure, see Antibodies: A Laboratory Manual, Cold Spring Harbor Laboratory, Eds. Harlow et al., 1988. One of skill in the art will recognize that each subunit structure, e.g., a CH, VH, CL, VL, CDR, and/or FR structure, comprises active fragments. For example, active fragments may consist of the portion of the VH, VL, or CDR subunit that binds the antigen, i.e., the antigen-binding fragment, or the portion of the CH subunit that binds to and/or activates an Fc receptor and/or complement.

Non-limiting examples of binding fragments encompassed within the term “antigen-specific antibody” used herein include: (i) an Fab fragment, a monovalent fragment consisting of the VL, VH, CL and CH1 domains; (ii) an F(ab′)2 fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment, which consists of a VH domain; and (vi) an isolated CDR. Furthermore, although the two domains of the Fv fragment, VL and VH, are coded for by separate genes, they may be recombinantly joined by a synthetic linker, creating a single protein chain in which the VL and VH domains pair to form monovalent molecules (known as single chain Fv (scFv)). The most commonly used linker is a 15-residue (Gly₄Ser)₃ peptide, but other linkers are also known in the art. Single chain antibodies are also intended to be encompassed within the terms “antibody or antigen binding protein,” or “antigen-binding fragment” of an antibody. The antibody can also be a polyclonal antibody, monoclonal antibody, chimeric antibody, antigen-binding fragment, Fc fragment, single chain antibodies, or any derivatives thereof.

Antibodies can be obtained using conventional techniques known to those skilled in the art, and the fragments are screened for utility in the same manner as intact antibodies. Antibody diversity is created by multiple germline genes encoding variable domains and a variety of somatic events. The somatic events include recombination of variable gene segments with diversity (D) and joining (J) gene segments to make a complete VH domain, and the recombination of variable and joining gene segments to make a complete VL domain. The recombination process itself is imprecise, resulting in the loss or addition of amino acids at the V(D)J junctions. These mechanisms of diversity occur in the developing B cell prior to antigen exposure. After antigenic stimulation, the expressed antibody genes in B cells undergo somatic mutation. Based on the estimated number of germline gene segments, the random recombination of these segments, and random VH-VL pairing, up to 1.6×10⁷ different antibodies may be produced (Fundamental Immunology, 3rd ed. (1993), ed. Paul, Raven Press, New York, N.Y.). When other processes that contribute to antibody diversity (such as somatic mutation) are taken into account, it is thought that upwards of 1×10¹⁰ different antibodies may be generated (Immunoglobulin Genes, 2nd ed. (1995), eds. Jonio et al., Academic Press, San Diego, Calif.). Because of the many processes involved in generating antibody diversity, it is unlikely that independently derived monoclonal antibodies with the same antigen specificity will have identical amino acid sequences.

Antibody or antigen binding protein molecules capable of specifically interacting with the antigens, epitopes, or other molecules described herein may be produced by methods well known to those skilled in the art. For example, monoclonal antibodies can be produced by generation of hybridomas in accordance with known methods. Hybridomas formed in this manner can then be screened using standard methods, such as enzyme-linked immunosorbent assay (ELISA) and Biacore analysis, to identify one or more hybridomas that produce an antibody that specifically interacts with a molecule or compound of interest. As an alternative to preparing monoclonal antibody-secreting hybridomas, a monoclonal antibody to a polypeptide of the present disclosure may be identified and isolated by screening a recombinant combinatorial immunoglobulin library (e.g., an antibody phage display library) with a polypeptide of the present disclosure to thereby isolate immunoglobulin library members that bind to the polypeptide. Techniques and commercially available kits for generating and screening phage display libraries are well known to those skilled in the art. Additionally, examples of methods and reagents particularly amenable for use in generating and screening antibody or antigen binding protein display libraries can be found in the literature.

Examples of chimeric antibodies include, but are not limited to, humanized antibodies. The antibodies described herein can also be human antibodies. In some embodiments, the capture reagent comprises a detection reagent. The detection reagent can be any reagent that can be used to detect the presence of the capture reagent binding to its specific binding partner. The capture reagent can comprise a detection reagent directly or the capture reagent can comprise a particle that comprises the detection reagent. In some embodiments, the capture reagent and/or particle comprises a color, colloidal gold, radioactive tag, fluorescent tag, or a chemiluminescent substrate. The particle can be, for example, a viral particle, a latex particle, a lipid particle, or a fluorescent particle.

In some embodiments, the antibody as described infra binds to the regions described herein or a peptide with at least 90, 95, or 99% homology or identity to the region. In some embodiments, the fragment of the regions described herein are 5-10 residues in length. In some embodiments, the fragment of the regions (e.g. epitope) described herein are 3-5 residues in length. The fragments are described based upon the length provided. In some embodiments, the epitope is about 3, 4, 5, 6, 7, 8, 9, 10, 11, 12, 13, 14, 15, or 20 residues in length.

In some embodiments, the sequence to which the antibody binds may include both nucleic acid and amino acid sequences. In some embodiments, the sequence to which the antibody binds may include sequences having at least about 60% homology with the disclosed sequences. In some embodiments, the sequence to which the antibody binds may have at least about 65%, about 70%, about 75%, about 80%, about 85%, about 90%, about 95%, about 97%, about 99%, about 99.8% homology with the disclosed sequences. In some embodiments, the sequences may be referred to as “mutant nucleic acids” or “mutant peptide sequences.”

The methods comprising the use of an antibody can be adapted to the method described in Ke Guo et al., Science Translational Medicine, (7 Sep. 2011) Vol. 3, Issue 99, p. 99ra85. This method can be used to generate an immune response (e.g. antibodies) against the proteins described herein even if the proteins are present on the inside of a cell.

Embodiments illustrating the method and materials used may be further understood by reference to the following non-limiting examples.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Microarray Analysis of Breast Cancer vs. Normal Breast Tissue

Microarray gene expression analysis. Total RNA was either sourced commercially or was prepared from cultured cells as follows. Total RNA was extracted directly from cells growing in 10 cm tissue culture dishes using Qiagen RNeasy mini kits according to the manufacturer's instructions. RNA concentrations were measured using a Beckman DU530 or Nanodrop spectrophotometer and RNA quality was determined by denaturing agarose gel electrophoresis or with an Agilent 2100 Bioanalyzer. Total RNA was linearly amplified and biotin-labeled using Illumina TotalPrep kits (Ambion), and cRNA was quality controlled using an Agilent 2100 Bioanalyzer. cRNA was hybridized to Illumina Human-HT-12 v4 BeadArrays, processed, and read using a BeadStation array reader according to the manufacturer's instructions (Illumina).

Raw data was imported into Genespring (Agilent Technologies) and percentile-shift normalized. To identify genes that are up-regulated in breast tumors (N=27) versus normal breast tissue (N=14) a fold-change analysis was performed in Genespring. Additionally, these genes were also analyzed for elevated expression relative to a diverse group of normal tissues (N=68) comprised of normal colon, cervix, uterus, ovary, fallopian tube, bone, skeletal muscle, skin, soft tissue, lung, kidney, esophagus, lymph node, thyroid, bladder, pancreas, liver, stomach, spinal chord, brain and salivary gland. Genes that are up-regulated in breast tumors versus normal breast tissue >2.1 fold are summarized in Table 1. Expression of the markers listed in Table 1 were elevated relative to normal tissue.

### Example 2

### Serum Detection of FSIP1

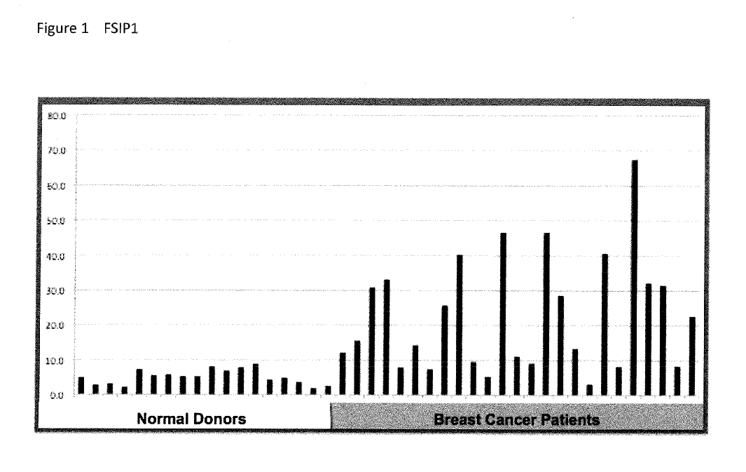

An ELISA for FSIP1 was performed using a commericially available kit according to the manufacturer's instructions (Proteogenix, Inc. Costa Mesa, Calif.). Samples included serum from subjects that did not have cancer (i.e. normal donors) and serum samples from patients diagnosed with breast cancer. Samples were analyzed using the Luminex® 200™ (Millipore Corp, Billerica, Mass.). The results shown in FIG. 1 represent the serum concentration (ng/ml) in each of the tested samples. The results show that FSIP1 is a good serum marker for breast cancer.

### Example 3

### Detection of FSIP1 in Breast Cancer Tissue

Paraffin embedded tissue sections of true normal breast (not adjacent normal to a tumor), fibroadenoma of the breast, and breast tumors (ductal carcinoma) were obtained from Asterand. The sections were dewaxed in xylene and rehydrated in cycles of ethanol (100%, 95%, 70%) followed by a wash in distilled water. Antigen retrieval was performed in epitope retrieval buffer (IHC World #IW-1100) by incubating the slides at 95±40 minutes using an IHC-Steamer Set (IHC World 41W-1102). Immunostaining was performed incubating over night at 4C with a polyclonal rabbit anti-human FSIP 1 antibody (Novus Biologicals #NBP1-56460) at a 1:100 dilution in IHC-Tek antibody dilution buffer (IHC World #IW-1001). The antibody was washed out by incubating the slides 30 minutes in IHC-Tek washing buffer (IHC World #IW-1201), with a change of buffer every 10 minutes. Subsequently the slides were incubated one hour with Alexa Fluor 594 goat anti-rabbit IgG (Life sciences #21207) at a 1:200 dilution in antibody dilution buffer. After this incubation time, the slides were washed as described above, and Vectashield mounting medium with DAPI was used to preserve the stained samples (Vector Laboratories #H-1200). Images were taken with an exposure time of 200 milliseconds using a Nikon Eclipse TE2000-U at a magnification of 10,000 and an X-Cite 120 fluorescence illumination system (Lumen Dynamics).

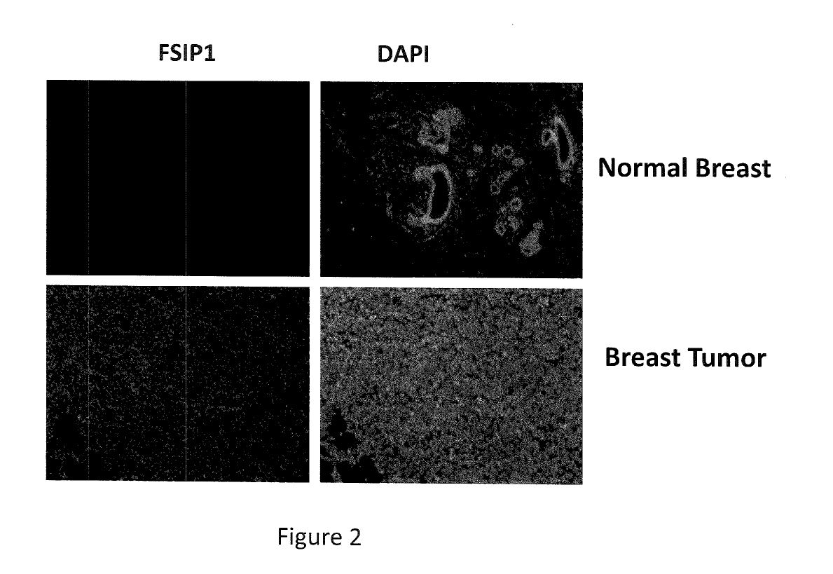

The results are shown in FIG. 2 and demonstrate that FSIP1 is strongly expressed in breast tumor tissue, but is not expressed in normal breast tissue.

### Example 4

### Serum Detection of Breast Cancer Markers

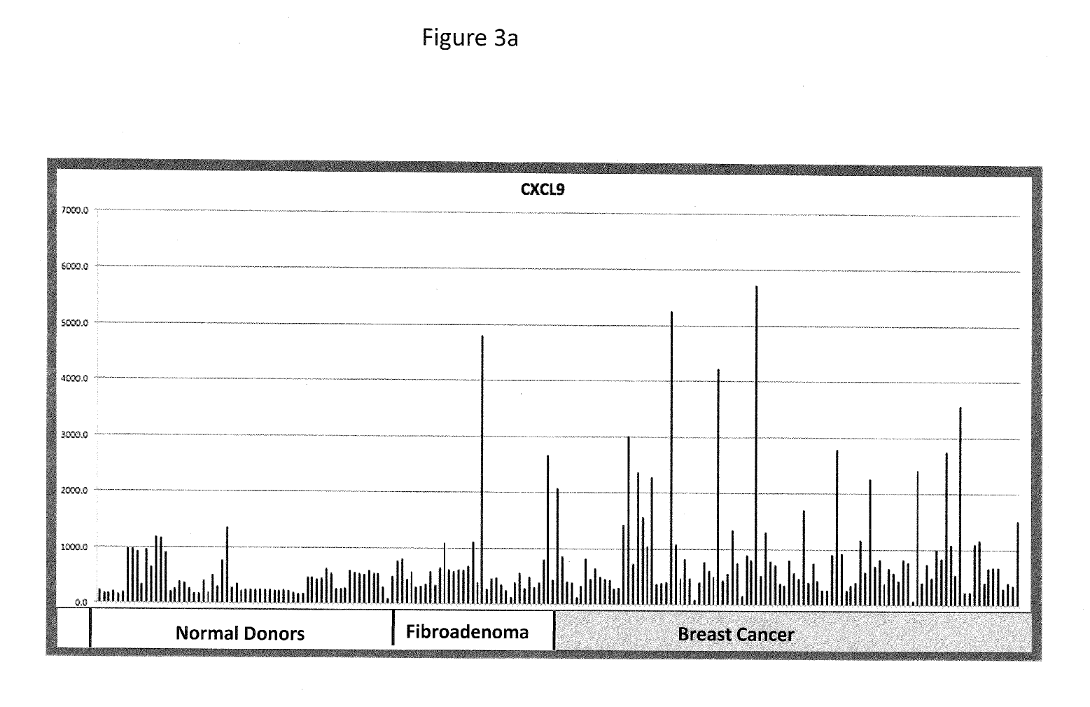

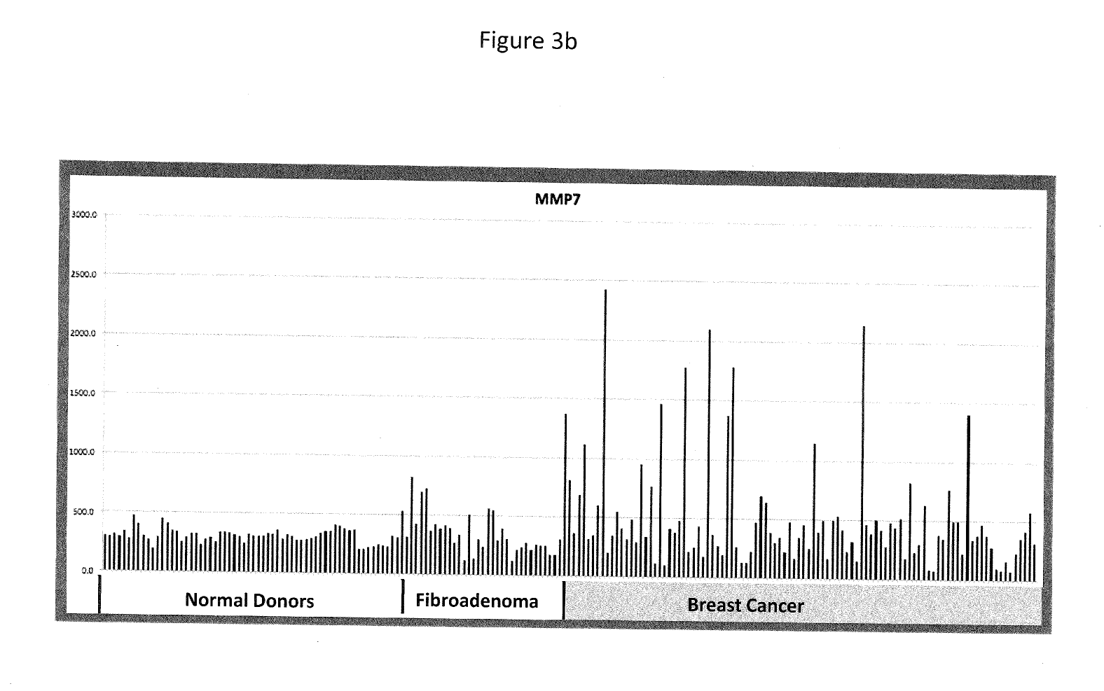

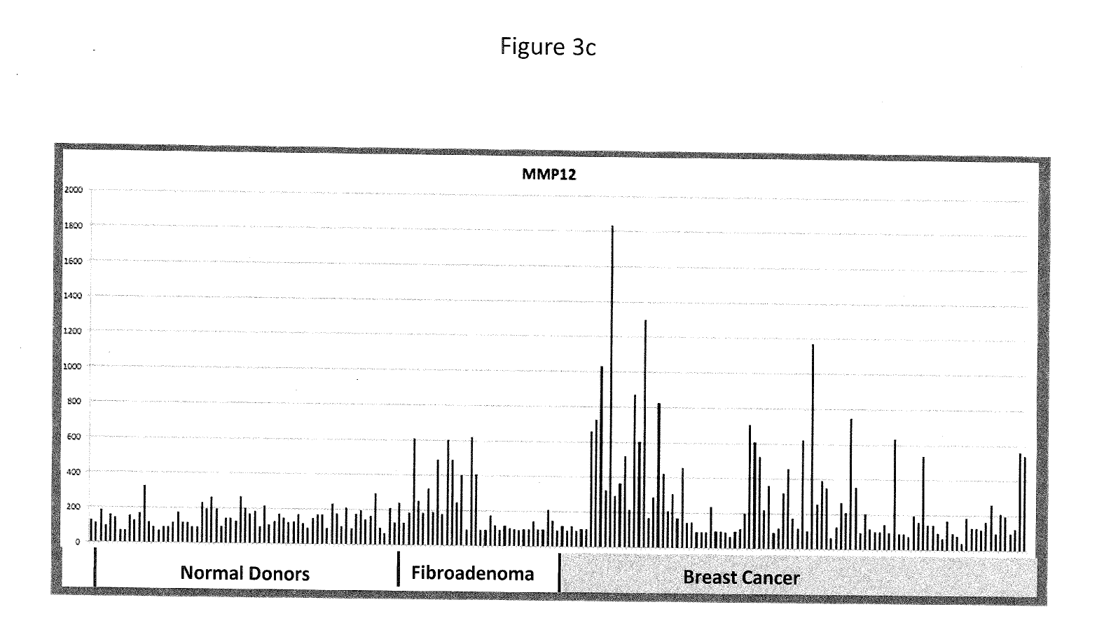

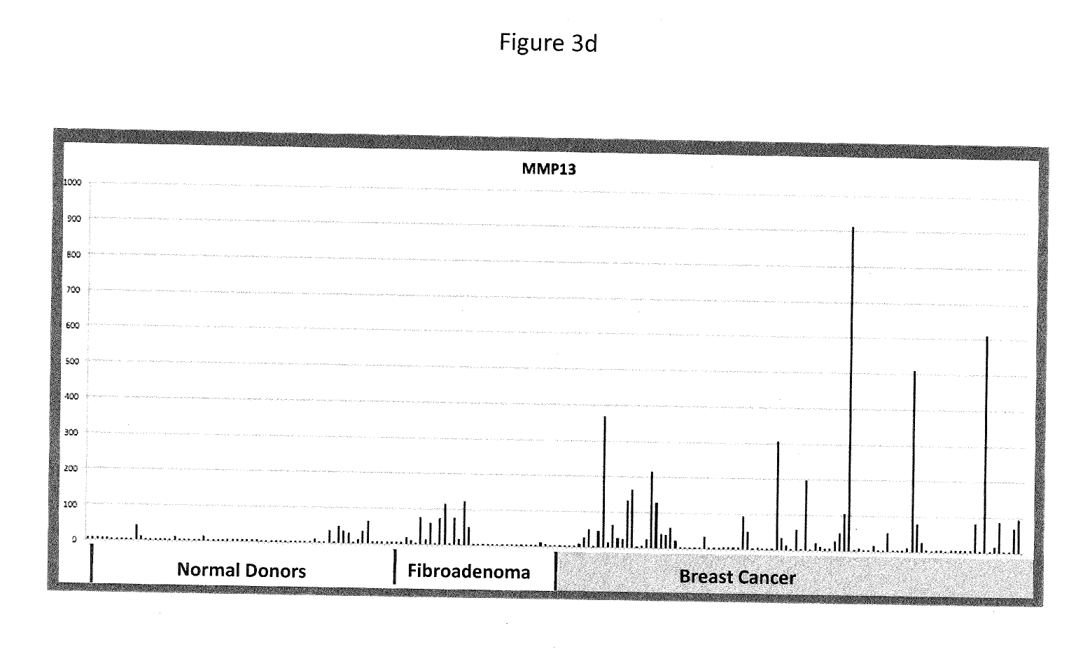

An ELISA for the markers CXCL9, MMP7, MMP12, and MMP13 was performed using a commericially available kit according to the manufacturer's instructions (Millipore Corp., Billerica, Mass.). Samples included serum from subjects that did not have cancer (i.e. normal donors) and serum samples from patients diagnosed with breast cancer. Samples were analyzed on theLuminex® 200™ (Millipore Corp, Billerica, Mass.). The results shown in FIG. 3 represent the serum concentration (ng/ml) in each of the tested samples. The results show that CXCL9, MMP7, MMP12, and MMP13 are good serum markers for breast cancer.

TABLE 1

| | | | | | Fold | | Fold | | | | |
|---|---|---|---|---|---|---|---|---|---|---|---|
| | | | | | change | Regulation | change | Regulation | | | |
| | | | | | ([Breast | ([Breast | ([Breast | ([Breast | Normal | | |
| | | | | | cancer] | cancer] | cancer] | cancer] | Breast | Breast | Normal |
| | | | | | vs | vs | vs | vs | Tissue | tumors | Tissues |
| Illumina | | | | | [Breast | [Breast | [Normal | [Normal | Averaged | Averaged | Averaged |
| Probe_Id | Definition | Accession | Probe_Sequence | Symbol | Normal]) | Normal]) | Tissues]) | Tissues]) | (N = 14) | (N = 27) | (N = 68) |
| ILMN_16559 [symbol] | Homo | NM_005940. [symbol] | CAGGTCTTGGT | MMP11 | 7.0 | up | 5.9 | up | −0.349 | 2.449 | −0.107 |
| | sapien | | AGGTG [symbol] | | | | | | | | |
| ILMN_16776 [symbol] | Homo | NM_000095. [symbol] | AGAGGACTATG | COMP | 5.7 | up | 4.3 | up | 0.182 | 2.683 | 0.587 |
| | sapien | | AGACC [symbol] | | | | | | | | |
| ILMN_21087 [symbol] | Homo | NM_001958. [symbol] | AGCGCTCGCC | EEF1A2 | 5.2 | up | 2.7 | up | −0.130 | 2.254 | 0.842 |
| | sapien | | ACGCTC [symbol] | | | | | | | | |
| ILMN_16727 [symbol] | Homo | NM_000493. [symbol] | CCCCTAAAAT | COL10A1 | 4.9 | up | 5.0 | up | −0.003 | 2.284 | −0.032 |
| | sapien | | ATTTCTG [symbol] | | | | | | | | |
| ILMN_16902 [symbol] | Homo | NM_014141. [symbol] | TTTTCAGTGA | CNTNAP2 | 4.3 | up | 2.8 | up | 0.391 | 2.503 | 1.004 |
| | sapien | | GCGTGG [symbol] | | | | | | | | |
| ILMN_16743 [symbol] | Homo | NM_002653. [symbol] | GGCCCGCGCC | PITX1 | 4.1 | up | 2.9 | up | −1.367 | 0.672 | −0.851 |
| | sapien | | CCTGTT [symbol] | | | | | | | | |
| ILMN_16514 [symbol] | Homo | NM_138720. [symbol] | TGTGGATCCC | HIST1H28D | 3.8 | up | 6.4 | up | 0.119 | 2.031 | −0.638 |
| | sapien | | ACCCAA [symbol] | | | | | | | | |
| ILMN_17453 [symbol] | Homo | NM_002416. [symbol] | TGATIGGTGC | CXCL9 | 3.6 | up | 3.1 | up | 0.593 | 2.431 | 0.799 |
| | sapien | | CCAGTT [symbol] | | | | | | | | |
| ILMN_17895 [symbol] | Homo | NM_001854. [symbol] | GGTGCCACCA | COL11A1 | 3.6 | up | 3.7 | up | −0.114 | 1.716 | −0.162 |
| | sapien | | ACCCAT [symbol] | | | | | | | | |
| I1MN_23477 [symbol] | Homo | NM_022872. [symbol] | AACTCGGTGG | IFI6 | 3.5 | up | 4.3 | up | 0.134 | 1.961 | −0.136 |
| | sapien | | CTGCCT [symbol] | | | | | | | | |
| ILMN_17125 [symbol] | Homo | NM_002483. [symbol] | CTAGGTGAGC | CEACAM6 | 3.4 | up | 2.6 | up | 0.590 | 2.353 | 0.992 |
| | sapien | | GCATTG [symbol] | | | | | | | | |
| ILMN_17511 [symbol] | Homo | NM_003543. [symbol] | CGCACTCTTT | HIST1H4H | 3.4 | up | 4.2 | up | 0.202 | 1.963 | −0.110 |
| | sapien | | ACGGGCTT [symbol] | | | | | | | | |
| ILMN_18012 [symbol] | Homo | NM_005980. [symbol] | AATGATGCCC | S100P | 3.4 | up | 2.0 | up | 0.242 | 1.997 | 1.001 |
| | sapien | | TGGAGA [symbol] | | | | | | | | |
| ILMN_17250 [symbol] | Homo | NM_138455. [symbol] | AGGACTTTGT | CTHRC1 | 3.3 | up | 5.5 | up | −0.362 | 1.372 | −1.099 |
| | sapien | | GAAGGA [symbol] | | | | | | | | |
| ILMN_17586 [symbol] | Homo | NM_138720. [symbol] | CTATTAACGC | HIST1H28D | 3.3 | up | 4.0 | up | −0.084 | 1.642 | −0.353 |
| | sapien | | TACGATC [symbol] | | | | | | | | |
| ILMN_17917 [symbol] | Homo | NM_001565. [symbol] | GACTTCCACT | CXCL10 | 3.3 | up | 4.1 | up | 0.731 | 2.452 | 0.402 |
| | sapien | | GCCATCC [symbol] | | | | | | | | |
| ILMN_17231 [symbol] | Homo | NM_022965. [symbol] | TCACCCAAAC | FGFR3 | 3.3 | up | 1.7 | up | −0.181 | 1.537 | 0.790 |
| | sapien | | CGGCAG [symbol] | | | | | | | | |
| ILMN_32429 [symbol] | Homo | NM_001040. [symbol] | CTACATGGCT | HIST2H2AA4 | 3.2 | up | 4.0 | up | 0.072 | 1.764 | −0.241 |
| | sapien | | GCGGT [symbol] | | | | | | | | |
| ILMN_20540 [symbol] | Homo | NM_005101. [symbol] | ACCTGAAGCA | ISG15 | 3.2 | up | 3.9 | up | −0.486 | 1.203 | −0.749 |
| | sapien | | GCAAGT [symbol] | | | | | | | | |
| ILMN_16590 [symbol] | Homo | NM_003516. [symbol] | CGACTTTCCC | HIST2H2AA3 | 3.2 | up | 3.9 | up | 0.021 | 1.708 | −0.265 |
| | sapien | | GATCGCC [symbol] | | | | | | | | |
| ILMN_16569 [symbol] | Homo | NM_001311. [symbol] | GCCGAGAGCC | CRIP1 | 3.2 | up | 6.2 | up | 0.917 | 2.599 | −0.040 |
| | sapien | | ACACTT [symbol] | | | | | | | | |
| ILMN_23010 [symbol] | Homo | NM_181800. [symbol] | GATAGTCCCT | UBE2C | 3.1 | up | 4.9 | up | −2.323 | −0.681 | −2.972 |
| | sapien | | TGAACA [symbol] | | | | | | | | |
| ILMN_21444 [symbol] | Homo | NM_003516. [symbol] | GGCCCGCGTC | HIST2H2AA3 | 3.1 | up | 3.9 | up | 0.184 | 1.802 | −0.156 |
| | sapien | | TCGAAG [symbol] | | | | | | | | |
| ILMN_16854 [symbol] | Homo | NM 020351. [symbol] | GAGACCGGGT | COL8A1 | 3.1 | up | 2.9 | up | −0.692 | 0.921 | −0.615 |
| | sapien | | GTTCCT [symbol] | | | | | | | | |
| ILMN_16785 [symbol] | Homo | NM_000125. [symbol] | GCTGTGCAC | ESR1 | 3.1 | up | 10.4 | up | 2.518 | 4.128 | 0.743 |
| | sapien | | CCTAGAA [symbol] | | | | | | | | |
| ILMN_17689 [symbol] | Homo | NM_003517. [symbol] | CGCCCAGGGC | HIST2H2AC | 3.0 | up | 3.7 | up | −0.030 | 1.573 | −0.317 |
| | sapien | | GGCGTT [symbol] | | | | | | | | |
| ILMN_16962 [symbol] | PREDICTED: | NM_939163. [symbol] | ATCCTTCCTT | FLI23152 | 3.0 | up | 3.8 | up | 0.508 | 2.098 | 0.159 |
| | [symbol] | | TACTCC [symbol] | | | | | | | | |
| ILMN_16860 [symbol] | Homo | NM_001067. [symbol] | TTTCAGCTCTT | TOP2A | 3.0 | up | 5.0 | up | −2.218 | −0.653 | −2.983 |
| | sapien | | GACCTG [symbol] | | | | | | | | |
| ILMN_16698 [symbol] | Homo | NM_014399. [symbol] | GTGGCACCTG | TSPAN13 | 2.9 | up | 5.0 | up | 0.503 | 2.055 | −0.272 |
| | sapien | | GAATTT [symbol] | | | | | | | | |
| ILMN_23310 [symbol] | Homo | NM_206938. [symbol] | CAGTGCTCAT | MS4A7 | 2.9 | up | 4.1 | up | 1.357 | 2.883 | 0.860 |
| | sapien | | TTTGAGA [symbol] | | | | | | | | |
| ILMN_18833 [symbol] | AGENCOURT | NM_001203. [symbol] | ACAGGCCTTT | BMPR18 | 2.9 | up | 1.6 | up | −0.372 | 1.142 | 0.452 |
| | [symbol] | | GACCTC [symbol] | | | | | | | | |
| ILMN 21305 [symbol] | Homo | NM_014399. [symbol] | TACCTTCAGC | TSPAN13 | 2.8 | up | 3.9 | up | 0.504 | 1.991 | 0.035 |
| | sapien | | CTCCATC [symbol] | | | | | | | | |
| ILMN_18141 [symbol] | Homo | NM_006408. [symbol] | GGGTTTAGGA | AGR2 | 2.8 | up | 5.2 | up | 1.534 | 3.018 | 0.632 |
| | sapien | | AACGT [symbol] | | | | | | | | |
| ILMN_17917 [symbol] | Homo | NM_005086. [symbol] | TCCTCCCCAC | TUBB3 | 2.8 | up | 2.0 | up | −2.048 | −0.582 | −1.610 |
| | sapien | | CTAGGCC [symbol] | | | | | | | | |
| ILMN_16513 [symbol] | Homo | NM_100582. [symbol] | GTCAGCTGGA | SPP1 | 2.8 | up | 3.0 | up | 0.788 | 2.251 | 0.668 |
| | sapien | | TGACCA [symbol] | | | | | | | | |
| ILMN_17224 [symbol] | Homo | NM_003225. [symbol] | TCCTGGTGTC | TFF1 | 2.7 | up | 9.0 | up | 2.156 | 3.579 | 0.405 |
| | sapien | | ACGCCC [symbol] | | | | | | | | |
| ILMN_20660 [symbol] | Homo | NM_178840. [symbol] | GATCCGCTAA | C1orf64 | 2.7 | up | 7.1 | up | 1.843 | 3.264 | 0.428 |
| | sapien | | GGGGCA [symbol] | | | | | | | | |
| ILMN_22859 [symbol] | Homo | NM_014736. [symbol] | AGGAGGGAAC | KIAA0101 | 2.6 | up | 4.7 | up | −1.953 | −0.550 | −2.783 |
| | sapien | | CCCGTT [symbol] | | | | | | | | |
| ILMN_16901 [symbol] | Homo | NM_007315. [symbol] | TACTCCAGGC | STAT1 | 2.6 | up | 2.6 | up | −0.644 | 0.743 | −0.652 |
| | sapien | | CAAAGG [symbol] | | | | | | | | |
| ILMN_17666 [symbol] | Homo | NM_004446. [symbol] | GCCATCGTGT | FOXA1 | 2.6 | up | 20.2 | up | 3.651 | 5.041 | 0.702 |
| | sapien | | GCTTGTT [symbol] | | | | | | | | |
| ILMN_22067 [symbol] | Homo | NM_001711. [symbol] | TCCAGCGCAA | BGN | 2.6 | up | 3.0 | up | −0.198 | 1.187 | −0.414 |
| | sapien | | GGAGGC [symbol] | | | | | | | | |
| ILMN_24123 [symbol] | Homo | NM_057735. [symbol] | GCTGTGGCTC | CCNE2 | 2.6 | up | 2.7 | up | −0.761 | 0.620 | −0.809 |
| | sapien | | CTTCCTA [symbol] | | | | | | | | |
| ILMN_22032 [symbol] | Homo | NM_002030. [symbol] | CATGTCTGGA | FPR3 | 2.6 | up | 2.8 | up | 0.575 | 1.956 | 0.476 |
| | sapien | | CCTCAG [symbol] | | | | | | | | |
| ILMN_17147 [symbol] | Homo | NM_181803. [symbol] | CCCTCATGAA | UBE2C | 2.6 | up | 3.3 | up | −1.980 | −0.603 | −2.341 |
| | sapien | | CCCAAC [symbol] | | | | | | | | |
| ILMN_24066 [symbol] | Homo | NM_002051. [symbol] | GCAGCCTGTG | GATA3 | 2.6 | up | 8.0 | up | 1.727 | 3.103 | 0.111 |
| | sapien | | CTGAGG [symbol] | | | | | | | | |
| ILMN_33078 [symbol] | Homo | NM_006408. [symbol] | GGCCTTGAGA | AGR2 | 2.6 | up | 9.1 | up | 2.972 | 4.345 | 1.166 |
| | sapien | | CTTGAA [symbol] | | | | | | | | |
| ILMN_16880 [symbol] | PREDICTED: | XM_0011272 [symbol] | TTGGTTTTGA | NAT1 | 2.6 | up | 3.2 | up | 0.045 | 1.414 | −0.247 |
| | [symbol] | | GACCACC [symbol] | | | | | | | | |
| ILMN_18080 [symbol] | Homo | NM_017843. [symbol] | GGCTTTGCAG | BCAS4 | 2.6 | up | 2.7 | up | −0.381 | 0.976 | −0.442 |
| | sapien | | GGGCTC [symbol] | | | | | | | | |
| ILMN_17169 [symbol] | Homo | NM_152597. [symbol] | GGTGGTCACT | FSIP1 | 2.6 | up | 2.9 | up | 0.138 | 1.489 | −0.028 |
| | sapien | | GGGAA [symbol] | | | | | | | | |
| ILMN_17038 [symbol] | Homo | NM_015130. [symbol] | GTATGCTCTA | T8C1D9 | 2.5 | up | 5.6 | up | 1.235 | 2.579 | 0.093 |
| | sapien | | TTTCCTG [symbol] | | | | | | | | |
| ILMN_21435 [symbol] | Homo | NM_012319. [symbol] | AGTTTCAGTA | SLC39A6 | 2.5 | up | 5.4 | up | 0.941 | 2.283 | −0.144 |
| | sapien | | GGTCAT [symbol] | | | | | | | | |
| ILMN_22176 [symbol] | Homo | NM_003568. [symbol] | CGCTGTACTT | ANXA9 | 2.5 | up | 2.7 | up | 0.284 | 1.625 | 0.202 |
| | sapien | | TGCTGAC [symbol] | | | | | | | | |
| ILMN_16925 [symbol] | PREDICTED: | XR_017364.1 | GTTCTGATTT | LOC653381 | 2.5 | up | 2.9 | up | −0.268 | 1.064 | −0.448 |
| | [symbol] | | TCTTGTA [symbol] | | | | | | | | |
| ILMN_23744 [symbol] | Homo | NM_001040 [symbol] | GGTCACTGAT | SPP1 | 2.5 | up | 3.0 | up | 0.871 | 2.202 | 0.621 |
| | sapien | | TTTCCCA [symbol] | | | | | | | | |
| ILMN_20722 [symbol] | Homo | NM_001827. [symbol] | CCAACAGAGT | CKS2 | 2.5 | up | 3.3 | up | −1.670 | −0.340 | −2.080 |
| | sapien | | CTAGGC [symbol] | | | | | | | | |
| ILMN_16913 [symbol] | Homo | NM_139266. [symbol] | CGCCATCACA | STAT1 | 2.5 | up | 2.3 | up | −0.686 | 0.643 | −0.584 |
| | sapien | | GCTGAA [symbol] | | | | | | | | |
| ILMN_16740 [symbol] | Homo | NM_016817. [symbol] | GAGCTCTGGG | OAS2 | 2.5 | up | 3.5 | up | 0.359 | 1.685 | −0.138 |
| | sapien | | CCCTTC [symbol] | | | | | | | | |
| ILMN_17320 [symbol] | Homo | NM_003528. [symbol] | CACTTACCAC | HIST2H28E | 2.5 | up | 2.7 | up | 0.371 | 1.692 | 0.268 |
| | sapien | | CTCATTT [symbol] | | | | | | | | |
| ILMN_20660 [symbol] | Homo | NM_001298.1 | AACCCCACAG | HLA-DR86 | 2.5 | up | 1.9 | up | 0.753 | 2.072 | 1.160 |
| | sapien | | CCTTGA [symbol] | | | | | | | | |
| ILMN_21175 [symbol] | Homo | NM_138455. [symbol] | CAGATTACCC | CTHRC1 | 2.5 | up | 2.5 | up | −0.307 | 1.003 | −0.296 |
| | sapien | | AAAAGG [symbol] | | | | | | | | |
| ILMN_23310 [symbol] | Homo | NM_206938. [symbol] | CCTCTCAACA | MS4A7 | 2.5 | up | 2.6 | up | 0.713 | 2.023 | 0.665 |
| | sapien | | CACAGC [symbol] | | | | | | | | |
| ILMN_17023 [symbol] | Homo | NM_015170. [symbol] | CCTCACTGAG | SULF1 | 2.5 | up | 3.1 | up | 0.516 | 1.822 | 0.176 |
| | sapien | | TCATCAC [symbol] | | | | | | | | |
| ILMN_17597 [symbol] | Homo | NM_206833. [symbol] | CCCTCCCATG | CTXN1 | 2.5 | up | 2.7 | up | −0.500 | 0.796 | −0.652 |
| | sapien | | CCGTTCC [symbol] | | | | | | | | |
| ILMN_18060 [symbol] | Homo | NM_903258. [symbol] | TGACATCAGC | TK1 | 2.4 | up | 3.7 | up | −1.219 | 0.073 | −1.829 |
| | sapien | | CTGCTTC [symbol] | | | | | | | | |
| ILMN_17092 [symbol] | Homo | NM_018962. [symbol] | TAGGGAGTAG | DSCR6 | 2.4 | up | 2.6 | up | −0.039 | 1.252 | −0.153 |
| | sapien | | AACCGT [symbol] | | | | | | | | |
| ILMN_17699 [symbol] | Homo | NM_030674. [symbol] | CTCTGGTCTG | SLC38A1 | 2.4 | up | 3.0 | up | −0.494 | 0.793 | −0.779 |
| | sapien | | AGTCAAC [symbol] | | | | | | | | |
| ILMN_23255 [symbol] | Homo | NM_017843. [symbol] | CTTCTGTTGC | BCAS4 | 2.4 | up | 2.7 | up | −0.326 | 0.958 | −0.477 |
| | sapien | | CTCCAGA [symbol] | | | | | | | | |
| ILMN_17574 [symbol] | Homo | NM_005319. [symbol] | GTGGCTAAGA | HIST1H1C | 2.4 | up | 2.6 | up | −0.019 | 1.256 | −0.103 |
| | sapien | | GCCCAA [symbol] | | | | | | | | |
| ILMN_17114 [symbol] | Homo | NM_014176. [symbol] | GCCAGCCTTC | UBE2T | 2.4 | up | 2.6 | up | −1.306 | −0.043 | −1.446 |
| | sapien | | CTCAAG [symbol] | | | | | | | | |
| ILMN_17170 [symbol] | Homo | NM_006645. [symbol] | GGCTCCCAAG | STARD10 | 2.4 | up | 3.3 | up | 0.886 | 2.147 | 0.418 |
| | sapien | | GCCATG [symbol] | | | | | | | | |
| ILMN_24023 [symbol] | Homo | NM_020351. [symbol] | CCGTGATGTA | COL8A1 | 2.4 | up | 1.7 | up | −0.625 | 0.634 | −0.152 |
| | sapien | | CACGTA [symbol] | | | | | | | | |
| ILMN_32396 [symbol] | PREDICTED: | XM_0017245 [symbol] | ATGCTAACTA | LOC1001335 [symbol] | 2.4 | up | 1.8 | up | 0.236 | 1.494 | 0.676 |
| | [symbol] | | GTTACG [symbol] | | | | | | | | |
| ILMN_20412 [symbol] | Homo | NM_003129. [symbol] | GTCAGAACCT | SQLE | 2.4 | up | 2.3 | up | −1.364 | −0.108 | −1.335 |
| | sapien | | TGGATT [symbol] | | | | | | | | |
| ILMN_23640 [symbol] | Homo | NM_004207. [symbol] | CACCCCTGGA | SLC16A3 | 2.4 | up | 1.7 | up | −2.136 | −0.893 | −1.622 |
| | sapien | | AGATGG [symbol] | | | | | | | | |
| ILMN_17241 [symbol] | Homo | NM_003655. [symbol] | GAAATGTATT | CBX4 | 2.4 | up | 2.6 | up | −0.249 | 0.984 | −0.394 |
| | sapien | | GTTTGA [symbol] | | | | | | | | |
| ILMN_17503 [symbol] | Homo | NM_012319. [symbol] | GACTTTGCTG | SLC39A6 | 2.3 | up | 5.7 | up | 0.984 | 2.213 | −0.302 |
| | sapien | | TTCTACT [symbol] | | | | | | | | |
| ILMN_17480 [symbol] | Homo | NM_002573. [symbol] | GCTGGAGCCC | PAFAH183 | 2.3 | up | 4.3 | up | −0.763 | 0.462 | −1.640 |
| | sapien | | GCACCC [symbol] | | | | | | | | |
| ILMN_16584 [symbol] | Homo | NM_020826. [symbol] | CTGGGCACAG | SYT13 | 2.3 | up | 1.6 | up | 0.111 | 1.330 | 0.653 |
| | sapien | | AGAATC [symbol] | | | | | | | | |
| ILMN_17752 [symbol] | Homo | NM_001025. [symbol] | GTAGATTCCC | AFF3 | 2.3 | up | 3.6 | up | 1.505 | 7.709 | 0.856 |
| | sapien | | AAGAGA [symbol] | | | | | | | | |
| ILMN_16873 [symbol] | Homo | NM_022873. [symbol] | TGCGCCGACG | IFI6 | 2.3 | up | 2.9 | up | 0.169 | 1.370 | −0.147 |
| | sapien | | ATGCCC [symbol] | | | | | | | | |
| ILMN_22207 [symbol] | Homo | NM_005752. [symbol] | CTCCTGGTGG | CLEC3A | 2.3 | up | 1.8 | up | −0.248 | 0.952 | 0.068 |
| | sapien | | GACTTG [symbol] | | | | | | | | |
| ILMN_17860 [symbol] | Homo | NM_001048. [symbol] | CCAGTTCTTC | UHRF1 | 2.3 | up | 2.9 | up | −1.292 | −0.094 | −1.644 |
| | sapien | | CTGACAC [symbol] | | | | | | | | |
| ILMN_17926 [symbol] | Homo | NM_003512. [symbol] | TCTGGACGTG | HIST1H2AC | 2.3 | up | 2.9 | up | 0.248 | 1.442 | −0.076 |
| | sapien | | GTAAGC [symbol] | | | | | | | | |
| ILMN_18151 [symbol] | Homo | NM_018136. [symbol] | CACAAATCCC | ASPM | 2.3 | up | 2.2 | up | −1.723 | −0.533 | −1.654 |
| | sapien | | CTGCAA [symbol] | | | | | | | | |
| ILMN_17267 [symbol] | Homo | NM_018454. [symbol] | CTAGGCTTTG | NUSAP1 | 2.3 | up | 3.1 | up | −1.747 | −0.559 | −2.181 |
| | sapien | | CTTAGTA [symbol] | | | | | | | | |
| ILMN_23885 [symbol] | Homo | NM_033255. [symbol] | GGGAGTCACT | EPSTI1 | 2.3 | up | 2.3 | up | −0.457 | 0.728 | −0.445 |
| | sapien | | TGATG [symbol] | | | | | | | | |
| ILMN_16686 [symbol] | Homo | NM_020853. [symbol] | TTCTTGTGAA | KIAA1467 | 2.3 | up | 2.9 | up | 0.432 | 1.606 | 0.092 |
| | sapien | | ATGTGTC [symbol] | | | | | | | | |
| ILMN_16661 [symbol] | Homo | NM_005368. [symbol] | CTGGGAACCG | MB | 2.2 | up | 3.4 | up | 1.417 | 2.578 | 0.803 |
| | sapien | | GGAGTT [symbol] | | | | | | | | |
| ILMN_17534 [symbol] | Homo | NM_001898. [symbol] | ATCCAGGTGT | CST1 | 2.2 | up | 1.9 | up | 0.191 | 1.351 | 0.438 |
| | sapien | | CAAGAA [symbol] | | | | | | | | |
| ILMN_17961 [symbol] | Homo | NM_080593. [symbol] | CCCACTGGGG | HIST1H28K | 2.2 | up | 3.4 | up | −0.081 | 1.076 | −0.707 |
| | sapien | | GGTTGG [symbol] | | | | | | | | |
| ILMN_21638 [symbol] | Homo | NM_032532. [symbol] | TCCAGGAATA | FNDC1 | 2.2 | up | 2.3 | up | 0.812 | 1.952 | 0.749 |
| | sapien | | GCATAT [symbol] | | | | | | | | |
| ILMN_17676 [symbol] | Homo | NM_203463. [symbol] | CACAGGTCCA | LASS6 | 2.2 | up | 2.5 | up | −0.143 | 0.992 | −0.355 |
| | sapien | | TGAAAG [symbol] | | | | | | | | |
| ILMN_17947 [symbol] | Homo | NM_016818. [symbol] | CAGCACTTGT | ABCG1 | 2.2 | up | 1.8 | up | −0.018 | 1.115 | 0.269 |
| | sapien | | GAAGGA [symbol] | | | | | | | | |
| ILMN_17239 [symbol] | Homo | NM_006820. [symbol] | GTGGGCTAAG | IFI44L | 2.2 | up | 2.4 | up | 0.626 | 1.756 | 0.499 |
| | sapien | | ATAGGT [symbol] | | | | | | | | |
| ILMN_16999 [symbol] | Homo | NM_138278. [symbol] | GCCCTTTTTC | BNIPL | 2.2 | up | 5.6 | up | 1.971 | 3.090 | 0.601 |
| | sapien | | CTTCTAA [symbol] | | | | | | | | |
| ILMN_17392 [symbol] | PREDICTED: | XM_937958. [symbol] | GCGCCATCTT | LOC648879 | 2.2 | up | 3.4 | up | 0.656 | 1.773 | −0.013 |
| | [symbol] | | GCCCTG [symbol] | | | | | | | | |
| ILMN_20427 [symbol] | Homo | NM_004219. [symbol] | CTCCACCATG | PTTG1 | 2.2 | up | 2.1 | up | −2.203 | −1.087 | −2.123 |
| | sapien | | GGAATCC [symbol] | | | | | | | | |
| ILMN_17891 [symbol] | Homo | NM_173633. [symbol] | CGGGGGCCTT | TMEM145 | 2.2 | up | 1.5 | up | −0.302 | 0.811 | 0.208 |
| | sapien | | CCCTCG [symbol] | | | | | | | | |
| ILMN_22692 [symbol] | Homo | NM_021800. [symbol] | GGAAGAGCAA | DNAJC12 | 2.2 | up | 4.1 | up | 1.106 | 2.218 | 0.198 |
| | sapien | | GACTG [symbol] | | | | | | | | |
| ILMN_17135 [symbol] | Homo | NM_012261. [symbol] | TGTCTTGGGA | C20orf103 | 2.2 | up | 1.7 | up | 0.437 | 1.548 | 0.768 |
| | sapien | | ATGTTT [symbol] | | | | | | | | |
| ILMN_17278 [symbol] | Homo | NM_115415. [symbol] | GAGTCTGTTT | 8RP44 | 2.2 | up | 2.1 | up | −0.289 | 0.819 | −0.225 |
| | sapien | | GCTGTTA [symbol] | | | | | | | | |
| ILMN 16607 [symbol] | Homo | NM_152341. [symbol] | CTTCCATCTG | PAQR4 | 2.2 | up | 2.5 | up | −0.694 | 0.410 | −0.913 |
| | sapien | | GCTGCA [symbol] | | | | | | | | |
| ILMN_16835 [symbol] | Homo | NM_201222. [symbol] | ATCCAGGCGG | MAGED2 | 2.1 | up | 3.2 | up | 0A35 | 1.537 | −0.142 |
| | sapien | | GAGCAC [symbol] | | | | | | | | |
| ILMN_18054 [symbol] | Homo | NM_032413. [symbol] | GAAGAGTTGC | C15orf48 | 2.1 | up | 2.5 | up | 0.862 | 1.963 | 0.643 |
| | sapien | | AAAATG [symbol] | | | | | | | | |
| ILMN_16518 [symbol] | Homo | NM_006317. [symbol] | CAGACAGAGC | BASP1 | 2.1 | up | 1.2 | up | −1.595 | −0.497 | −0.715 |
| | sapien | | CCACTT [symbol] | | | | | | | | |
| ILMN 16663 [symbol] | Homo | NM_005192. [symbol] | CGCAGATGGA | CDKN3 | 2.1 | up | 2.3 | up | −1.727 | −0.638 | −1.833 |
| | sapien | | GGGAC [symbol] | | | | | | | | |
| ILMN_16606 [symbol] | Homo | NM_006868. [symbol] | CCCTGTAGTC | RAB31 | 2.1 | up | 1.6 | up | −0.826 | 0.260 | −0.382 |
| | sapien | | CAGTGG [symbol] | | | | | | | | |
| ILMN_21043 [symbol] | Homo | NM_000089. [symbol] | GATCCACATT | COL1A2 | 2.1 | up | 6.8 | up | 1.024 | 2.108 | −0.665 |
| | sapien | | GTTAGT [symbol] | | | | | | | | |
| ILMN_16901 [symbol] | Homo | NM_001878. [symbol] | CCAAGTCAGC | CRABP2 | 2.1 | up | 9.0 | up | 1.931 | 3.013 | −0.149 |
| | sapien | | AGTCCT [symbol] | | | | | | | | |
| ILMN_16640 [symbol] | Homo | NM_138793. [symbol] | TGCCTGGCTG | CANT1 | 2.1 | up | 2.9 | up | 0.037 | 1.118 | −0.441 |
| | sapien | | TCTCCTT [symbol] | | | | | | | | |
| ILMN_23895 [symbol] | Homo | NM_002734. [symbol] | CTGCTCTAGA | PRKAR1A | 2.1 | up | 1.4 | up | −0.613 | 0.463 | 0.011 |
| | sapien | | AAGTAT [symbol] | | | | | | | | |
| ILMN_23829 [symbol] | Homo | NM_001218. [symbol] | GCTGCATGGC | CA12 | 2.1 | up | 4.7 | up | 1.008 | 2.084 | −0.145 |
| | sapien | | AGATGC [symbol] | | | | | | | | |
| ILMN_17346 [symbol] | Homo | NM_032532. [symbol] | GGCCATTCTG | FNDC1 | 2.1 | up | 2.1 | up | 0.733 | 1.806 | 0.740 |
| | sapien | | GTCATC [symbol] | | | | | | | | |
| ILMN_18030 [symbol] | Homo | NM_021800. [symbol] | GTTGGCACCT | DNAIC12 | 2.1 | up | 3.4 | up | 0.923 | 1.995 | 0.223 |
| | sapien | | TCGTTTC [symbol] | | | | | | | | |
| ILMN_21064 [symbol] | Homo | NM_024613. [symbol] | GTGCGGATTT | PLEKHF2 | 2.1 | up | 2.9 | up | 0.191 | 1.263 | −0.296 |
| | sapien | | GTGACT [symbol] | | | | | | | | |

[symbol]

indicates data missing or illegible when filed

TABLE 2

| | | | Regu- | | Regu- | | | | | |
|---|---|---|---|---|---|---|---|---|---|---|
| | | FC | lation | FC | lation | | | | | |
| | | ([Breast | ([Breast | ([Breast | ([Breast | | | | | |
| | | tumor] | tumor] | tumor] | tumor] | | | | | |
| | | vs | vs | vs | vs | | Normal | | Normal | |
| | | [Normal | [Normal | [normal | [normal | Breast | Blood | Normal | Cell | normal |
| Symbol | Accession | Breast]) | Breast]) | tissue]) | tissue]) | tumor | Cells | Breast | Line | tissue |
| MTL5 | NM_004923.3 | 2.1 | up | 2.6 | up | 1.3899549 | 0.45535946 | 0.3429471 | −0.0980946 | 0.01354783 |
| FAM196A | NM_001039762.1 | 1.9 | up | 1.6 | up | 0.8250304 | 0.06369641 | −0.0773228 | 0.06693005 | 0.13210669 |
| ANKRD30B | XR_016169.1 | 1.7 | up | 2.1 | up | 1.1138475 | 0.17779769 | 0.3537288 | −0.0161494 | 0.05151437 |
| SYCP2 | NM_014258.2 | 1.5 | up | 2.3 | up | 1.2090596 | 0.13592702 | 0.5840468 | −0.0786644 | 0.00554682 |
| KIFC2 | NM_145754.2 | 2.1 | up | 2.3 | up | 1.13396 | 0.1500082 | 0.07397024 | −0.7366369 | −0.0613983 |
| AURKA | NM_198434.1 | 2.1 | up | 2.5 | up | −0.4924355 | −1.0322843 | −1.5559984 | 0.52166224 | −1.8011446 |
| CCNB2 | NM_004701.2 | 2.2 | up | 2.7 | up | −0.7434611 | −0.8714966 | −1.8844538 | 0.6876289 | −2.1561859 |
| SUSD3 | NM_145006.2 | 2.2 | up | 3.4 | up | 2.1707983 | 1.5274293 | 1.0108994 | −0.0605537 | 0.41047648 |
| AKR7A3 | NM_012067.2 | 2.2 | up | 2.3 | up | 1.5286146 | −0.4608932 | 0.42194986 | 0.21606033 | 0.3255132 |
| DACH1 | NM_080760.3 | 2.1 | up | 2.1 | up | 1.690513 | 0.50327164 | 0.6309643 | 0.2191774 | 0.5872986 |
| MELK | NM_014791.2 | 2.2 | up | 2.3 | up | −0.5691218 | −0.8007338 | −1.7120414 | 0.75228095 | −1.7420491 |
| CLSTN2 | NM_022131.1 | 2.1 | up | 2.1 | up | 1.5553231 | −0.0475502 | 0.48450097 | 0.16923557 | 0.49766213 |
| VAV3 | NM_006113.4 | 2.0 | up | 4.3 | up | 2.4194808 | 0.91523755 | 1.4081693 | −0.30866 | 0.30872792 |
| PARP9 | NM_031458.1 | 2.1 | up | 2.3 | up | 1.0379184 | −0.0659555 | −0.0082068 | −0.2610467 | −0.1431574 |
| CDC20 | NM_001255.2 | 2.0 | up | 2.9 | up | −0.9165652 | −1.6705098 | −1.9469439 | 0.8152109 | −2.462041 |
| GFRA1 | NM_005264.3 | 2.1 | up | 2.6 | up | 1.5281755 | −0.0289959 | 0.47806576 | 0.14481325 | 0.15208387 |
| CEP55 | NM_018131.3 | 2.3 | up | 2.4 | up | −0.561767 | −1.1823134 | −1.7745 | 0.6531127 | −1.8362858 |
| TUBA1C | NM_032704.3 | 2.1 | up | 2.3 | up | −0.5396962 | −1.5870992 | −1.6219743 | 0.75124884 | −1.7462262 |
| MX1 | NM_002462.2 | 2.3 | up | 2.2 | up | 1.5304146 | 0.12022516 | 0.3466634 | −1.1049972 | 0.3807559 |
| SBK1 | NM_001024401.2 | 2.3 | up | 3.3 | up | 1.7881644 | 0.10731271 | 0.60385555 | −0.3908531 | 0.05418417 |
| OLR1 | NM_002543.3 | 2.2 | up | 2.9 | up | 1.9421784 | 0.41789207 | 0.8274854 | −0.267893 | 0.42048544 |
| LOC1001280 [symbol] | XM_001721625.1 | 2.4 | up | 2.1 | up | 1.1856675 | 0.12797125 | −0.0827344 | 0.03396525 | 0.1059223 |
| ZNF467 | NM_207336.1 | 2.0 | up | 2.7 | up | 1.464282 | 0.83677906 | 0.44599453 | 0.07023053 | 0.04314031 |
| MAL2 | NM_052886.2 | 2.2 | up | 4.6 | up | 2.1467836 | −2.3374686 | 0.99844676 | −1.488375 | −0.0501761 |
| IFIT1 | NM_001548.3 | 2.3 | up | 2.4 | up | 1.5407792 | −0.6786563 | 0.33485183 | −0.2722928 | 0.24819472 |
| TUFT1 | NM_020127.1 | 2.1 | up | 2.7 | up | 1.1315321 | −1.6126075 | 0.09555622 | −0.267251 | −0.2965135 |
| C2orf27A | NM_013310.3 | 2.0 | up | 2.0 | up | 0.9372299 | 0.0066674 | −0.0303777 | 0.11781777 | −0.0539879 |
| FLJ22184 | NM_001080403.1 | 2.0 | up | 3.3 | up | 1.9143692 | −0.3837198 | 0.9011822 | −0.1756829 | 0.18525231 |

[symbol]

indicates data missing or illegible when filed

TABLE 3

| Symbol | Alias | Probe_Id | Definition | Accession | Probe_Sequence |
|---|---|---|---|---|---|
| MTL5 | | ILMN_1661778 | Homo sapien | NM_004923.3 | AGATATTTCCCCAGAGGCACGCGAA |
| | | | | | CTGTCAGTCTTTCCTAAGGCCCCCG |
| FAM196A | 10orf141 | ILMN_2071682 | Homo sapien | NM_001039762.1 | GCAAGGTAAAACGCACAGGGTTCCC |
| | | | | | TTATGTAGATGTACACATGGCATTG |
| ANKRD30B | LOC642460 | ILMN_1672000 | PRE- | XR_016169.1 | AAGCCTACCTGTGGAAGGAAAGTTT |
| | | | DICTED: H | | CTCTTCCAAATAAAGCCTTAGAATT |
| SYCP2 | | ILMN_2095704 | Homo sapien | NM_014258.2 | GGATGAGAGGGAACCACTATAACAT |
| | | | | | GAGTCCAAGCCCAGAAGACTTCTGT |
| KIFC2 | | ILMN_1803018 | Homo sapien | NM_145754.2 | GTGACGGCTGGTGACTGATGGATGG |
| | | | | | GTAGTGGGCTGAGAAGAGGGGACTA |
| AURKA | | ILMN_2357438 | Homo sapien | NM_198434.1 | TCCTTTAGGAATGCTGTGTGTCTGT |
| | | | | | CCGGCACCCCGGTAGGCCTGATTGG |
| CCNB2 | | ILMN_1801939 | Homo sapien | NM_004701.2 | CAGCATGATCCCTCAGCTGAACTCA |
| | | | | | AAAGCCGTCAAAGACCTTGCCTCCC |
| SUSD3 | | ILMN_1785570 | Homo sapien | NM_145006.2 | GAATCAGCTTCCAGGTGTAGGGACC |
| | | | | | CCTTGAGGGGCCAAGCTGACATCCA |
| AKR7A3 | | ILMN_2145396 | Homo sapien | NM_012067.2 | CTTCCGCTAGGCCCATCGTTTCTCA |
| | | | | | GGCTGCCCAAGGCTCTTCTGTAACA |
| DACH1 | | ILMN_1755741 | Homo sapien | NM_080760.3 | GGTAGCCTAGTTATTTGAGCCTGGT |
| | | | | | TTCAATGTGAGAACCACGTTTACTG |
| MELK | | ILMN_2212909 | Homo sapien | NM_014791.2 | TGATGGATTCTTCCATCCTGCCGGA |
| | | | | | TGAGTGTGGGTGTGATACAGCCTAC |
| CLSTN2 | | ILMN_1731237 | Homo sapien | NM_022131.1 | TCAATGTGTATGCTCTGTCCCCATC |
| | | | | | CTTCACTCCTCCTCAAGCTCACACC |
| VAV3 | | ILMN_1657679 | Homo sapien | NM_006113.4 | GTTGCATTTCATGGGCCTGGGGGTT |
| | | | | | TCCTAGCAGAGGATATTGGAGCCCC |
| PARP9 | | ILMN_1731224 | Homo sapien | NM_031458.1 | CCTTTGCACAGCATCCTTGGAGGGG |
| | | | | | ATTCGCAAGTGGCAGCCCTGTTGAT |
| CDC20 | | ILMN_1663390 | Homo sapien | NM_001255.2 | AAGGCCAGTGCAGCCAAAAGCAGCC |
| | | | | | TCATCCACCAAGGCATCCGCTGAAG |
| GFRA1 | | ILMN_2334359 | Homo sapien | NM_005264.3 | TCCCTGAACGACACTCTCCTAATCC |
| | | | | | TAAGCCTTACCTGAGTGAGAAGCCC |
| CEP55 | | ILMN_1747016 | Homo sapien | NM_018131.3 | GCTGTGTTCCCCAACTCTGTTCTGC |
| | | | | | GCACGAAACAGTATCTGTTTGAGGC |
| TUBA1C | | ILMN_3251341 | Homo sapien | NM_032704.3 | AGAAGGATTATGAGGAGGTTGGAGC |
| | | | | | AGATAGTGCTGACGGAGAGGATGAG |
| MX1 | | ILMN_1662358 | Homo sapien | NM_002462.2 | ACCCAGTGTTTTAGGAGCATGAGTG |
| | | | | | CCGTGTGTGTGCGTCCTGTCGGAGC |
| SBK1 | | ILMN_1728298 | Homo sapien | NM_001024401.2 | CAGAGCCCCAGCCCCTCATGTCTTG |
| | | | | | CCGCCCTTCCTCCATGTGTTTGTAA |
| OLR1 | | ILMN_1723035 | Homo sapien | NM_002543.3 | CCCTCAAAGTGTCACAATGCTCCTC |
| | | | | | CTGATGACTCCTCCCCAGAAAACCA |
| LOC100128098 | | ILMN_3184978 | PRE- | XM_001721625.1 | GGGCAGATTCTGGCTGCCTTTTAAT |
| | | | DICTED: H | | TTCGTCCTICACCTGATATCTGTGC |
| ZNF467 | | ILMN_1779015 | Homo sapien | NM_207336.1 | AAAGCAGATATTTCCCGGACCCAGC |
| | | | | | GCGGCCTCAACCAGGGCAGGAAAGA |
| MAL2 | | ILMN_1770653 | Homo sapien | NM_052886.2 | CTGGCTGAAGCATCCCCTTGGAGTG |
| | | | | | CCATGTATAAGTTGGGCTATTAGAG |
| IFIT1 | | ILMN_1707695 | Homo sapien | NM_001548.3 | TGAATGAAGCCCTGGAGTACTATGA |
| | | | | | GCGGGCCCTGAGACTGGCTGCTGAC |
| TUFT1 | | ILMN_1781374 | Homo sapien | NM_020127.1 | ACGCAGCCAGAATCCTTAAGTCTGT |
| | | | | | GTGTTTCTGTGTCTCAAGACTGGGC |
| C2orf27A | | ILMN_1684726 | Homo sapien | NM_013310.3 | CCAACATGCTCTAATGCTTCAGATT |
| | | | | | CAAGTGCTTTTTCCACTGTTTCCCC |
| FLJ22184 | | ILMN_2387471 | Homo sapien | NM_001080403.1 | TTGCCCACCCCGCACAGCCTGAGTT |
| | | | | | TGCAATAAAACTGGGACACTGGGAC |

1. A method of detecting breast cancer in a subject comprising a) obtaining a sample from a subject; b) contacting the sample obtained from the subject with a plurality of agents that detect expression of a plurality of markers listed in Table 1 and/or Table 2; c) contacting a non-cancerous cell with the plurality of agents from b); and d) comparing the expression level of one or more of the markers listed in Table 1 and/or Table 2 in the sample obtained from the subject with the expression level of one or more of the markers listed in Table 1 and or Table 2 in the non-cancerous cell, wherein a higher level of expression of the plurality of the markers listed in Table 1 and/or Table 2 in the sample compared to the non-cancerous cell indicates that the subject has breast cancer.

2. The method of claim 1 wherein the agent is a nucleic acide.

3. The method of claim 1, wherein the nucleic acid is DNA.

4. The method of claim 3, wherein the DNA comprises 5-30 nucloetides.

5. The method of claim 1 wherein the subject is a human.

6. The method of claim, wherein the agent is an antibody.

7. A kit comprising a plurality of agents that bind to a plurality of markers found in Table 1 and/or Table 2.

8. The kit of claim 7, wherein the plurality of agents are nucleic acid molecules.

9. The kit of claim 8, wherein the nucleic acid molecules are DNA.

10. The kit of claim 7, wherein the plurality of agents are protein molecules.

11. The kit of claim 10, wherein the protein molecules are antibodies.

12. A kit comprising a plurality of agents that bind to the markers found in Table 1 and/or Table 2.

13. The kit of claim 12, wherein the plurality of agents are nucleic acid molecules.

14. The kit of claim 13, wherein the nucleic acid molecules are DNA.

15. The kit of claim 12, wherein the plurality of agents are protein molecules.

16. The kit of claim 15, wherein the protein molecules are antibodies.

17. The kit of claim 7, wherein the plurality of markers is chosen from FSIP, CXCL9, MMP7, MMP13, and MMP12.

18. The kit of claim 7, wherein the plurality of markers are FSIP, CXCL9, MMP7, MMP13, and MMP12.

19. The kit of claim 17, wherein the plurality of agents are antibodies.

20. The kit of claim 17, wherein the plurality of agents are oligonucleotides.
